(19) United States
(12) Patent Application Publication
(10) Pub. No.: US 2023/0340481 A1
(43) Pub. Date: Oct. 26, 2023

(54) SYSTEMS AND METHODS FOR TRANSPOSING CARGO NUCLEOTIDE SEQUENCES

(71) Applicant: METAGENOMI, INC., Emeryville, CA (US)

(72) Inventors: Brian THOMAS, Emeryville, CA (US); Christopher BROWN, Emeryville, CA (US); Daniela S.A. GOLTSMAN, Emeryville, CA (US); Cristina BUTTERFIELD, Emeryville, CA (US); Lisa ALEXANDER, Emeryville, CA (US); Jason LIU, Emeryville, CA (US)

(21) Appl. No.: 18/188,231

(22) Filed: Mar. 22, 2023

Related U.S. Application Data
(63) Continuation of application No. PCT/US2021/047196, filed on Aug. 23, 2021.
(60) Provisional application No. 63/082,983, filed on Sep. 24, 2020.
(60) Provisional application No. 63/187,290, filed on May 11, 2021.
(60) Provisional application No. 63/232,578, filed on Aug. 12, 2021.

(51) Int. Cl. C12N 15/113; C12N 9/22

(52) U.S. Cl. CPC C12N 15/113; C12N 9/22; C12N 2320/10; C12N 2330/50

(57) ABSTRACT

The present disclosure provides systems and methods for transposing a cargo nucleotide sequence to a target nucleic acid site. These systems and methods may comprise a first double-stranded nucleic acid comprising the cargo nucleotide sequence, wherein the cargo nucleotide sequence is configured to interact with a recombinase complex, a cas effector complex comprising a cas effector and at least one engineered guide polynucleotide configured to hybridize to the target nucleic acid site, and the recombinase complex wherein said recombinase complex is configured to recruit the cargo nucleotide to the target nucleic acid site.

### RELATED APPLICATIONS

This application is a continuation of International Application No. PCT/US2021/047196, filed Aug. 23, 2021, entitled “SYSTEMS AND METHODS FOR TRANSPOSING CARGO NUCLEOTIDE SEQUENCES”, which claims the benefit of U.S. Provisional Application No. 63/082,983, filed on Sep. 24, 2020, entitled “SYSTEMS AND METHODS FOR TRANSPOSING CARGO NUCLEOTIDE SEQUENCES”, U.S. Provisional Application No. 63/187,290, filed May 11, 2021, entitled “SYSTEMS AND METHODS FOR TRANSPOSING CARGO NUCLEOTIDE SEQUENCES”, and U.S. Provisional Application No. 63/232,578, filed Aug. 12, 2021, entitled “SYSTEMS AND METHODS FOR TRANSPOSING CARGO NUCLEOTIDE SEQUENCES”, each of which is incorporated by reference in its entirety herein.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SEQUENCE LISTING

The instant application contains a Sequence Listing which has been submitted electronically in XML format and is hereby incorporated by reference in its entirety. Said XML copy, created on Mar. 3, 2023, is named 55921-714_302_SL.xml and is 220,882 bytes in size.

### SUMMARY

In some aspects, the present disclosure provides for a system for transposing a cargo nucleotide sequence to a target nucleic acid site comprising: a first double-stranded nucleic acid comprising a cargo nucleotide sequence configured to interact with a Tn7 type transposase complex; a Cas effector complex comprising a class II, type V Cas effector and an engineered guide polynucleotide configured to hybridize to said target nucleotide sequence; and a Tn7 type transposase complex configured to bind said Cas effector complex, wherein said Tn7 type transposase complex comprises a TnsB subunit. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 3′ of said target nucleic acid site. In some embodiments, said PAM sequence is located 5′ of said target nucleic acid site. In some embodiments, said engineered guide polynucleotide is configured to bind said class II, type V Cas effector. In some embodiments, said class II, type V Cas effector comprises a polypeptide comprising a sequence having at least 80% identity to SEQ ID NO: 1, 12, 16, 20-30, 64, or 80-85, or a variant thereof. In some embodiments, said TnsB subunit comprises a polypeptide having a sequence having at least 80% identity to SEQ ID NO: 2, 13, 17, or 65, or a variant thereof. In some embodiments, said Tn7 type transposase complex comprises at least one or at least two three polypeptide(s) comprising a sequence having at least 80% identity to any one of SEQ ID NOs: 3-4, 14-15, 18-19, or 66-67, or a variant thereof. In some embodiments, said engineered guide polynucleotide comprises a sequence comprising at least about 46-80 consecutive nucleotides having at least 80% identity to any one of SEQ ID NOs: 5-6, 32-33, 94-95, or 104-105, or a variant thereof. In some embodiments, said engineered guide polynucleotide comprises a sequence having at least 80% sequence identity to non-degenerate nucleotides of any one of SEQ ID NOs: 106, 107, 108, 5, 45-63, 68-75, or 96-103, or a variant thereof. In some embodiments, said left-hand recombinase sequence comprises a sequence having at least 80% identity to SEQ ID NO: 9, 11, 36-38, 76, or 78, or a variant thereof. In some embodiments, said right-hand recombinase sequence comprises a sequence having at least 80% identity to SEQ ID NO: 8, 10, 39-44, 77, 79, or 93, or a variant thereof. In some embodiments, said class II, type V Cas effector and said Tn7 type transposase complex are encoded by polynucleotide sequences comprising fewer than about 10 kilobases.

In some aspects, the present disclosure provides for a method for transposing a cargo nucleotide sequence to a target nucleic acid site comprising a target nucleotide sequence comprising expressing the system of any of the aspects or embodiments described herein within a cell or introducing the system of any of the aspects or embodiments described herein to a cell.

In some aspects, the present disclosure provides for a method for transposing a cargo nucleotide sequence to a target nucleic acid site, comprising contacting a first double-stranded nucleic acid comprising said cargo nucleotide sequence with: a Cas effector complex comprising a class II, type V Cas effector and at least one engineered guide polynucleotide configured to hybridize to said target nucleotide sequence; a Tn7 type transposase complex configured to bind said Cas effector complex, wherein said Tn7 type transposase complex comprises a TnsB subunit; and a second double-stranded nucleic acid comprising said target nucleic acid site. In some embodiments, said cargo nucleotide sequence is flanked by a left-hand transposase recognition sequence and a right-hand transposase recognition sequence. In some embodiments, the system further comprises a PAM sequence compatible with said Cas effector complex adjacent to said target nucleic acid site. In some embodiments, said PAM sequence is located 3′ of said target nucleic acid site. In some embodiments, said engineered guide polynucleotide is configured to bind said class II, type V Cas effector. In some embodiments, said class II, type V Cas effector comprises a polypeptide comprising a sequence having at least 80% identity to SEQ ID NO: 1, 12, 16, 20-30, 64, or 80-85, or a variant thereof. In some embodiments, said TnsB subunit comprises a polypeptide having a sequence having at least 80% identity to SEQ ID NO: 2, 13, 17, or 65, or a variant thereof. In some embodiments, said Tn7 type transposase complex comprises at least one or at least two polypeptide(s) comprising a sequence having at least 80% identity to any one of SEQ ID NOs: 3-4, 14-15, 18-19, or 66-67, or a variant thereof. In some embodiments, said engineered guide polynucleotide comprises a sequence comprising at least about 46-80 consecutive nucleotides having at least 80% identity to any one of SEQ ID NOs: 5-6, 32-33, 94-95, or 104-105, or a variant thereof. In some embodiments, said left-hand recombinase sequence comprises a sequence having at least 80% identity to SEQ ID NO: 9, 11, 36-38, 76, or 78, or a variant thereof. In some embodiments, said right-hand recombinase sequence comprises a sequence having at least 80% identity to SEQ ID NO: 8, 10, 39-44, 77, 79, or 93, or a variant thereof. In some embodiments, said class II, type V Cas effector and said Tn7 type transposase complex are encoded by polynucleotide sequences comprising fewer than about 10 kilobases.

In some aspects, the present disclosure provides for a system for transposing a cargo nucleotide sequence to a target nucleic acid site comprising: a first double-stranded nucleic acid comprising a cargo nucleotide sequence configured to interact with a Tn7 type transposase complex; a Cas effector complex comprising a class II, type V Cas effector and an engineered guide polynucleotide configured to hybridize to said target nucleotide sequence; and a Tn7 type transposase complex configured to bind said Cas effector complex, wherein said Tn7 type transposase complex comprises TnsB, TnsC, and TniQ components, wherein: (a) said class II, type V Cas effector comprises a polypeptide having a sequence having at least 80% sequence identity to any one of SEQ ID NO: 1, 12, 16, 20-30, 64, or 80-85, or a variant thereof or (b) said Tn7 type transposase complex comprises a TnsB, TnsC, or TniQ component having a sequence having at least 80% sequence identity to any one of SEQ ID NOs: 2-4, 13-15, 17-19, or 65-67, or a variant thereof. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said class II, type V Cas effector comprises a polypeptide having a sequence having at least 80% sequence identity to any one of SEQ ID NO: 1, 12, 16, 20-30, 64, or 80-85, or a variant thereof. In some embodiments, said Tn7 type transposase complex comprises a TnsB, TnsC, or TniQ component having a sequence having at least 80% sequence identity to any one of SEQ ID NOs: 2-4, 13-15, 17-19, or 65-67, or a variant thereof. In some embodiments, said class II, type V Cas effector is a Cas12k effector. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 5′ or 3′ of said target nucleic acid site. In some embodiments, said PAM sequence comprises SEQ ID NO:31. In some embodiments, said engineered guide polynucleotide is configured to bind said class II, type V Cas effector. In some embodiments, said engineered guide polynucleotide comprises a sequence comprising at least about 46-80 consecutive nucleotides having at least 80% identity to any one of SEQ ID NOs: 5-6, 32-33, 94-95, or 104-105, or a variant thereof. In some embodiments, said engineered guide polynucleotide comprises a sequence having at least 80% sequence identity to non-degenerate nucleotides of any one of SEQ ID NOs: 106, 107, 108, 5, 45-63, 68-75, or 96-103, or a variant thereof. In some embodiments, said left-hand recombinase sequence comprises a sequence having at least 80% identity to any one of SEQ ID NOs: 9, 11, 36-38, 76, or 78, or a variant thereof. In some embodiments, said right-hand recombinase sequence comprises a sequence having at least 80% identity to any one of SEQ ID NO: 8, 10, 39-44, 77, 79, or 93. In some embodiments, said class II, type V Cas effector and said Tn7 type transposase complex are encoded by polynucleotide sequences comprising fewer than about 10 kilobases. In some embodiments: (a) said class II, type V Cas effector comprises a sequence having at least 80% sequence identity to any one of SEQ ID NOs:1, 81, 82, 83, or 85, or a variant thereof; (b) said left-hand recombinase sequence comprises a sequence having at least 80% sequence identity to any one of SEQ ID NOs: 9, 11, 36, 37, or 38, or a variant thereof; (c) said right-hand recombinase sequence comprises a sequence having at least 80% identity to any one of SEQ ID NOs: 8, 39, 40, 41, 42, 43, 44, or 93, or a variant thereof; (d) said engineered guide polynucleotide: (i) comprises a sequence having at least 80% sequence identity to at least about 46-80 nucleotides of SEQ ID NO: 6, or a variant thereof; or (ii) comprises a sequence having at least 80% identity to the non-degenerate nucleotides of any one of SEQ ID NO: 5, 45-63, 68-75, or 96-103, or a variant thereof; (e) said TnsB, TnsC, and TniQ components comprise polypeptides having a sequence having at least 80% identity to SEQ ID NO: 2-4, or variants thereof; or (f) said PAM sequence comprises SEQ ID NO:31. In some embodiments: (a) said class II, type V Cas effector comprises a sequence having at least 80% sequence identity to SEQ ID NO:12, or a variant thereof; (b) said left-hand recombinase sequence comprises a sequence having at least 80% sequence identity to SEQ ID NO:76, or a variant thereof; (c) said right-hand recombinase sequence comprises a sequence having at least 80% identity to SEQ ID NO:77, or a variant thereof; (d) said engineered guide polynucleotide: (i) comprises a sequence having at least 80% sequence identity to at least about 46-80 nucleotides of SEQ ID NO: 32 or 104, or a variant thereof; or (ii) comprises a sequence having at least 80% identity to the non-degenerate nucleotides of any one of SEQ ID NO: 107 or 102, or a variant thereof; or (e) said TnsB, TnsC, and TniQ components comprise polypeptides having a sequence having at least 80% identity SEQ ID NO:13-15, or variants thereof. In some embodiments: (a) said class II, type V Cas effector comprises a sequence having at least 80% sequence identity to SEQ ID NO:16, or a variant thereof; (b) said left-hand recombinase sequence comprises a sequence having at least 80% sequence identity to SEQ ID NO:78, or a variant thereof; (c) said right-hand recombinase sequence comprises a sequence having at least 80% identity to SEQ ID NO:79, or a variant thereof; (d) said engineered guide polynucleotide: (i) comprises a sequence having at least 80% sequence identity to at least about 46-80 nucleotides of SEQ ID NO: 33 or 105, or a variant thereof; or (ii) comprises a sequence having at least 80% identity to the non-degenerate nucleotides of any one of SEQ ID NO: 108 or 103, or a variant thereof; or (e) said TnsB, TnsC, and TniQ components comprise polypeptides having a sequence having at least 80% identity SEQ ID NO: 17-19, or variants thereof.

In some aspects, the present disclosure provides for an engineered nuclease system comprising: an endonuclease comprising a RuvC domain, wherein said endonuclease is derived from an uncultivated microorganism, and wherein said endonuclease is a Class II, type V-K Cas effector having at least 80% identity to any one SEQ ID NO: 1, 12, 16, 20-30, 64, or 80-85, or a variant thereof; and an engineered guide RNA, wherein said engineered guide RNA is configured to form a complex with said endonuclease and said engineered guide RNA comprises a spacer sequence configured to hybridize to a target nucleic acid sequence. In some embodiments, said engineered guide polynucleotide comprises a sequence comprising at least about 46-80 consecutive nucleotides having at least 80% identity to any one of SEQ ID NOs: 5-6, 32-33, 94-95, or 104-105, or a variant thereof. In some embodiments, said engineered guide polynucleotide comprises a sequence having at least 80% identity to non-degenerate nucleotides of any one of SEQ ID NOs: 106, 107, 108, 5, 45-63, 68-75, or 96-103, or a variant thereof. In some embodiments, the system further comprises a PAM sequence compatible with said Cas effector complex adjacent to said target nucleic acid site. In some embodiments, said PAM sequence is located 5′ of said target nucleic acid site. In some embodiments, said PAM sequence comprises SEQ ID NO:31. In some embodiments: (a) said class II, type V-K Cas effector comprises a sequence having at least 80% sequence identity to any one of SEQ ID NOs:1, 81, 82, 83, or 85, or a variant thereof; (b) said left-hand recombinase sequence comprises a sequence having at least 80% sequence identity to any one of SEQ ID NOs: 9, 11, 36, 37, or 38, or a variant thereof; (c) said right-hand recombinase sequence comprises a sequence having at least 80% identity to any one of SEQ ID NOs: 8, 39, 40, 41, 42, 43, 44, or 93, or a variant thereof; (d) said engineered guide polynucleotide: (i) comprises a sequence having at least 80% sequence identity to at least about 46-80 nucleotides of SEQ ID NO: 6, or a variant thereof, or (ii) comprises a sequence having at least 80% identity to the non-degenerate nucleotides of any one of SEQ ID NO: 5, 45-63, 68-75, or 96-103, or a variant thereof; (e) said TnsB, TnsC, and TniQ components comprise polypeptides having a sequence having at least 80% identity to SEQ ID NO: 2-4, or variants thereof, or (f) said PAM sequence comprises SEQ ID NO:31.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts typical organizations of CRISPR/Cas loci of different classes and types.

FIG. 2 depicts the architecture of a natural Class II Type II crRNA/tracrRNA pair shown e.g. for Cas9, compared to a hybrid sgRNA wherein the crRNA and tracrRNA are joined.

FIG. 3 depicts the two pathways found in Tn7 and Tn7-like elements.

FIG. 4A and FIG. 4B depict the genomic context of a Type V Tn7 CAST of the family MG64. FIG. 4A Top: The MG64-1 CAST system consists of a CRISPR array (CRISPR repeats), a Type V nuclease, and three predicted transposase protein sequences. A tracrRNA was predicted in the intergenic region between the CAST effector and CRISPR array. Bottom: Multiple sequence alignment of the catalytic domain of transposase TnsB. The catalytic residues are indicated by boxes. FIG. 4B shows the two transposon ends predicted for the MG64-1 CAST system.

FIG. 5A and FIG. 5B depict predicted structures of corresponding sgRNAs of CAST systems described herein. FIG. 5A (left) shows the predicted MG64-1 tracrRNA and crRNA duplex complexes at the repeat-antirepeat stem. Loop was truncated and a tetraloop of GAAA was added to the stem loop structure to produce the designed sgRNA shown in FIG. 5B (right).

FIG. 6 depicts the results of a transposition reaction targeted to a plasmid Library consisting of NNNNNNNN at the 5′ of the target spacer sequence. Reaction #1 indicates the presence of the target Library, #2 shows presence of Donor fragments in both transposition reactions, #3-5 shows sg specific PCR band that corresponds to proper transposition reactions.

FIGS. 7A-7D depict the results of Sanger sequencing. FIG. 7A shows Sanger sequencing of the donor target junction on the transposon Left End (LE) in LE-closer-to-PAM transposition reactions. Expected sequence is at the top of the panel, with a predicted transposition event 61 bp away from the PAM. Top chromatogram is sequencing result that begins from within the donor fragment. Clear signal is seen on the right end up until the donor/target junction (dotted line). This denotes a mix of transposition products. The bottom chromatogram of the panel is sequencing from the target to the donor/target junction. The signal from the left is clear signal until the point of junction. FIG. 7B shows Sanger sequencing of the donor target junction on the transposon Right End (RE) in LE-closer-to-PAM products. Expected sequence is at the top of the panel, with a predicted transposition event 61 bp away from the PAM. Top chromatogram is sequencing result than begin from within the donor fragment. Clear signal is seen on the left end up until the donor/target junction (dotted line). FIG. 7C is a close up of the PAM library. FIG. 7D is the SeqLogo analysis on NGS of the LE-closer-to-PAM events which indicates a very strong preference for NGTN in the PAM motif.

FIG. 8 depicts a phylogenetic gene tree of Cas12k effector sequences. The tree was inferred from a multiple sequence alignment of 64 Cas12k sequences recovered here (orange and black branches) and 229 reference Cas12k sequences from public databases (grey branches). Orange branches indicate Cas12k effectors with confirmed association with CAST transposon components.

FIG. 9 shows MG64 family CRISPR repeat alignment. Cas12k CAST CRISPR repeats contain a conserved motif 5′-GNNGGNNTGAAAG-3′. In MG64-1, short repeat-antirepeats (RAR) within the CRISPR repeat motif align with the tracrRNA. MG64 RAR motifs appear to define the start and end of the tracrRNA (5′ end: RAR1 (TTTC); 3′ end: RAR2 (CCNNC)).

FIG. 10A and FIG. 10B depicts secondary structure predicted from folding the CRISPR repeat+tracrRNA for MG64 systems.

FIG. 11A depicts the MG64-3 CRISPR locus. The tracrRNA is encoded upstream from the CRISPR array, while the transposon end is encoded downstream (inner black box). A sequence corresponding to a partial 3′ CRISPR repeat and a partial spacer are encoded within the transposon (outer box). The self-matching spacer is encoded outside of the transposon end. FIG. 11B depicts tracrRNA sequence alignment for various CASTs provided herein. Alignment of tracrRNA sequences shows regions of conservation. In particular, the sequence “TGCTTTC” at sequence position 92-98 (top box) is suggested to be important for sgRNA tertiary structure and for a non-continuous repeat-anti-repeat pairing with the crRNA. We also suggest that the hairpin “CYCC(n6)GGRG” at positions 265-278 (bottom box) is important for function, possibly positioning the downstream sequence for crRNA pairing.

FIG. 12A depicts the predicted structure of MG64-1 sgRNA. FIG. 12B depicts the predicted structure of MG64-3 sgRNA. FIG. 12C depicts the predicted structure of MG64-5 sgRNA.

FIGS. 13A-13C depict PCR data which demonstrate that MG64-1 is active with sgRNA v2-1. Using the protocol described for In vitro targeted integrase activity, the effector protein and its TnsB, TnsC, and TniQ proteins were expressed in an in vitro transcription/translation system. After translation, the target DNA, cargo DNA, and sgRNA were added in reaction buffer. Integration was assayed by PCR across the target/donor junctions. FIG. 13A depicts a diagram illustrating the potential orientation of integrated donor DNA. PCR reactions 3, 4, 5, and 6 represent each integration ligation product depending on the orientation in which the donor was integrated at the target site. FIG. 13B depicts a gel image of PCR 4 (detecting the RE junction to the donor) of transposition showing: lane 1) apo (no sgRNA), lane 2) with sgRNA 1, and lane 3) with sgRNA v2-1. FIG. 13C depicts a gel image of PCR 5 (detecting the LE junction to the donor) of transposition showing: lane 1) apo (no sgRNA), lane 2) with sgRNA 1, and lane 3) with sgRNA v2-1.

FIG. 14 depicts PCR reaction 5 (LE proximal to PAM, top half of plot) and PCR reaction 4 (RE distal to PAM, bottom half of plot) plotted on the sequence and distance from the PAM for MG64-1. Analysis of the integration window indicates that 95% of the integrations that occur at the spacer PAM site are within a 10 bp window between 58 and 68 nucleotides away from the PAM. Differences in the integration distance between the distal and the proximal frequencies reflects the integration site duplication—a 3-5 base pair duplication as a result of staggered nuclease activity of the transposase upon integration.

FIG. 15 depicts the results of a colony PCR screen of Transposition Efficiency. After incubation, 18 colony forming units (CFUs) were visible on the plates; 8 on plate A (no IPTG, lanes labeled as A) and 10 on plate B (with 100 μM IPTG in recovery, lanes labeled as B). All 18 were analyzed by colony PCR, which gave a product band indicative of a successful transposition reaction (arrows).

FIG. 16 depicts sequencing results of select colony PCR products which confirm that they represent transposition events, as they span the junction between the LE and the PAM at the engineered target site, which is in the lacZ gene. The minimal LE sequence is indicated in blue at the top of the screen (min LE), while the target and PAM are indicated in grey. Some sequence variation is observed in the PCR products, but this variation is expected given that insertion can occur at variable distances upstream of the PAM.

FIGS. 17A-17H depict the results of testing of engineered single guides for 64-1 transposition activity. Black boxes are lanes not pertaining to this experiment. FIG. 17A depicts a gel image of PCR 4 (detecting the RE junction to the donor) of transposition: lane 1=apo (no sgRNA), lane 2=holo (+sgRNA), lane 3=sgRNA v1-1, lane 4=sgRNA v1-2, lane 5=sgRNA v1-3. FIG. 17B depicts a gel image of PCR 5 (detecting the LE junction to the donor) of transposition: lane 1=apo (no sgRNA), lane 2=holo (+sgRNA), lane 3=sgRNA v1-1, lane 4=sgRNA v1-2, lane 5=sgRNA v1-3. FIG. 17C depicts a gel image of PCR 4 (detecting the RE junction to the donor) of transposition: lane 1=apo (no sgRNA), lane 2=holo (+sgRNA), lane 3=sgRNA v1-4, lane 4=sgRNA v1-6, lane 5=sgRNA v1-7, lane 6=sgRNA v1-8, lane 7=sgRNA v1-9. FIG. 17D depicts a gel image of PCR 5 (detecting the LE junction to the donor) of transposition: lane 1=apo (no sgRNA), lane 2=holo (+sgRNA), lane 3=sgRNA v1-4, lane 4=sgRNA v1-6, lane 5=sgRNA v1-7, lane 6=sgRNA v1-8, lane 7=sgRNA v1-9. FIG. 17E depicts a gel image of PCR 4 (detecting the RE junction to the donor) of transposition: lane 1=apo (no sgRNA), lane 2=holo (+sgRNA), lane 3=sgRNA v1-5, lane 4=skip, lane 5=sgRNA v1-10. FIG. 17F depicts a gel image of PCR 5 (detecting the LE junction to the donor) of transposition: lane 1=apo (no sgRNA), lane 2=holo (+sgRNA), lane 3=sgRNA v1-5, lane 4=skip, lane 5=sgRNA v1-10. FIG. 17G depicts a gel image of PCR 4 (detecting the RE junction to the donor) of transposition: lane 1=apo (no sgRNA), lane 2=holo (+sgRNA), lane 3=sgRNAv1-17, lane 4=sgRNA v1-18, lane 5=skip, lane 6=sgRNA v1-19, lane 7=skip, lane 8=sgRNA v1-20. FIG. 17H depicts a gel image of PCR 5 (detecting the LE junction to the donor) of transposition: lane 1=apo (no sgRNA), lane 2=holo (+sgRNA), lane 3=sgRNAv1-17, lane 4=sgRNA v1-18, lane 5=skip, lane 6=sgRNA v1-19, lane 7=skip, lane 8=sgRNA v1-20

FIGS. 18A-18G depict the results of testing of engineered LE and RE for 64-1 transposition activity. Black boxes are lanes not pertaining to this experiment. FIG. 18A depicts a gel image of PCR 4 (detecting the RE junction to the donor) of transposition: lane 1=apo (no sgRNA), lane 2=holo (+sgRNA), lane 3=LE 86 bp, lane 4=LE 105 bp, lane 5=RE 196 bp, lane 6=RE 242 bp, lane 7=RE Internal deletion 50, lane 8=RE internal deletion 81. FIG. 18B depicts a gel image of PCR 5 (detecting the LE junction to the donor) of transposition: lane 1=apo (no sgRNA), lane 2=holo (+sgRNA), lane 3=LE 86 bp, lane 4=LE 105 bp, lane 5=RE 196 bp, lane 6=RE 242 bp, lane 7=RE Internal deletion 50, lane 8=RE internal deletion 81. FIG. 18C depicts a gel image of PCR 4 (detecting the RE junction to the donor) of transposition: lane 1=apo (no sgRNA), lane 2=holo (+sgRNA), lane 3=RE internal deletion 81 and 178 bp, lane 4=skip, lane 5=RE internal deletion 81 and 196 bp, lane 6=skip, lane 7=RE internal deletion 81 and 212 bp, lane 8=skip. FIG. 18D depicts a gel image of PCR 5 (detecting the LE junction to the donor) of transposition: lane 1=apo (no sgRNA), lane 2=holo (+sgRNA), lane 3=RE internal deletion 81 and 178 bp, lane 4=skip, lane 5=RE internal deletion 81 and 196 bp, lane 6=skip, lane 7=RE internal deletion 81 and 212 bp, lane 8=skip. FIG. 18E depicts a gel image of PCR 4 (detecting the RE junction to the donor) of transposition: lane 1=apo (no sgRNA), lane 2=holo (+sgRNA), lane 3=RE internal deletion 81 and 178 bp+LE 68 bp, lane 4=RE internal deletion 81 and 178 bp+LE 86 bp, lane 5=skip, lane 6=RE internal deletion 81 and 178 bp+LE 105 bp, lane 7=skip. FIG. 18F depicts a gel image of PCR 5 (detecting the LE junction to the donor) of transposition: lane 1=apo (no sgRNA), lane 2=holo (+sgRNA), lane 3=RE internal deletion 81 and 178 bp+LE 68 bp, lane 4=RE internal deletion 81 and 178 bp+LE 86 bp, lane 5=skip, lane 6=RE internal deletion 81 and 178 bp+LE 105 bp, lane 7=skip. FIG. 18G depicts a gel image of PCR 6 (detecting the RE junction to the donor) of transposition: lane 1=apo (no sgRNA), lane 2=holo (+sgRNA), lane 3=Obp overhang, lane 4=1 bp overhang, lane 5=2 bp overhang, lane 6=3 bp overhang, lane 7=5 bp overhang, lane 8=10 bp overhang.

FIGS. 19A-19J depict the results of testing of engineered CAST components with an NLS for transposition activity. Black boxes are lanes not pertaining to this experiment. FIG. 19A depicts a gel image of PCR 4 (detecting the RE junction to the donor) of transposition: lane 1=apo (no sgRNA), lane 2=holo (+sgRNA), lane 3=skip, lane 4=skip, lane 5=skip, lane 6=NLS-TnsB, lane 7=skip, lane 8=TnsB-NLS. FIG. 19B depicts a gel image of PCR 5 (detecting the LE junction to the donor) of transposition: lane 1=apo (no sgRNA), lane 2=holo (+sgRNA), lane 3=skip, lane 4=skip, lane 5=skip, lane 6=NLS-TnsB, lane 7=skip, lane 8=TnsB-NLS. FIG. 19C depicts a gel image of PCR 4 (detecting the RE junction to the donor) of transposition: lane 1=apo (no sgRNA), lane 2=holo (+sgRNA), lane 3=skip, lane 4=skip, lane 5=skip, lane 6=NLS-TniQ, lane 7=skip, lane 8=TniQ-NLS. FIG. 19D depicts a gel image of PCR 5 (detecting the LE junction to the donor) of transposition: lane 1=apo (no sgRNA), lane 2=holo (+sgRNA), lane 3=skip, lane 4=skip, lane 5=skip, lane 6=NLS-TniQ, lane 7=skip, lane 8=TniQ-NLS. FIG. 19E depicts a gel image of PCR 4 (detecting the RE junction to the donor) of transposition: lane 1=apo (no sgRNA), lane 2=holo (+sgRNA), lane 3=skip, lane 4=skip, lane 5=NLS-Cas12k, lane 6=Cas12k-NLS, lane 7=NLS-TnsC, lane 8=TnsC-NLS. FIG. 19F depicts a gel image of PCR 5 (detecting the LE junction to the donor) of transposition: lane 1=apo (no sgRNA), lane 2=holo (+sgRNA), lane 3=skip, lane 4=skip, lane 5=NLS-Cas12k, lane 6=Cas12k-NLS, lane 7=NLS-TnsC, lane 8=TnsC-NLS. FIG. 19G depicts a gel image of PCR 4 (detecting the RE junction to the donor) of transposition: lane 1=apo (no sgRNA), lane 2=holo (+sgRNA), lane 3=NLS-HA-TnsC, lane 4=NLS-TnsC-FLAG, lane 5=NLS-TnsC-HA, lane 6=NLS-TnsC-Myc, lane 7=NLS-FLAG-TnsC, lane 8=NLS-Myc-TnsC. FIG. 19H depicts a gel image of PCR 5 (detecting the LE junction to the donor) of transposition: lane 1=apo (no sgRNA), lane 2=holo (+sgRNA), lane 3=NLS-HA-TnsC, lane 4=NLS-TnsC-FLAG, lane 5=NLS-TnsC-HA, lane 6=NLS-TnsC-Myc, lane 7=NLS-FLAG-TnsC, lane 8=NLS-Myc-TnsC. FIG. 19I depicts a gel image of PCR 4 (detecting the RE junction to the donor) of transposition: lane 1=apo (no sgRNA), lane 2=holo (+sgRNA), lane 3=Cas 2x NLS apo (no sgRNA), lane 4=Cas 2x NLS holo (+sgRNA). FIG. 19J depicts a gel image of PCR 5 (detecting the LE junction to the donor) of transposition: lane 1=apo (no sgRNA), lane 2=holo (+sgRNA), lane 3=Cas 2x NLS apo (no sgRNA), lane 4=Cas 2x NLS holo (+sgRNA)

FIG. 20A and FIG. 20B depict engineered CAST-NLS acting as a single suite. All lanes have Cas12k-NLS and NLS-TniQ, TnsB, TnsC and sgRNA unless otherwise described. FIG. 20A depicts gel image of PCR 4 (detecting the RE junction to the donor) of transposition: lane 1=apo (no sgRNA), lane 2=holo (+sgRNA), lane 3=NLS-TnsB, lane 4=TnsB-NLS, lane 5=NLS-TnsB and NLS-TnsC, lane 6=TnsB-NLS and NLS-TnsC. FIG. 20B depicts gel image of PCR 5 (detecting the LE junction to the donor) of transposition: lane 1=apo (no sgRNA), lane 2=holo (+sgRNA), lane 3=NLS-TnsB, lane 4=TnsB-NLS, lane 5=NLS-TnsB and NLS-TnsC, lane 6=TnsB-NLS and NLS-TnsC.

FIGS. 21A-21H depict the results of testing of Cas Effector and TniQ protein fusion for transposition activity. FIG. 21A depicts a gel image of PCR 4 (detecting the RE junction to the donor) of transposition: lane 1=apo (no sgRNA) with Cas-TniQ fusion, lane 2=holo (+sgRNA) with Cas-TniQ fusion, lane 3=apo (no sgRNA) with TniQ-Cas fusion, lane 4=holo (+sgRNA) with TniQ-Cas fusion. FIG. 21B depicts a gel image of PCR 5 (detecting the LE junction to the donor) of transposition: lane 1=apo (no sgRNA) with Cas-TniQ fusion, lane 2=holo (+sgRNA) with Cas-TniQ fusion, lane 3=apo (no sgRNA) with TniQ-Cas fusion, lane 4=holo (+sgRNA) with TniQ-Cas fusion. FIG. 21C depicts a gel image of PCR 4 (detecting the RE junction to the donor) of transposition: lane 1=apo (no sgRNA) with TniQ-Cas fusion, lane 2=holo (+sgRNA) with TniQ-Cas fusion, lane 3=holo Cas alone, lane 4=apo (no sgRNA) with TniQ-48 Linker-Cas fusion, lane 5=holo (+sgRNA) with TniQ-48 Linker-Cas fusion, lane 6=apo (no sgRNA) with TniQ-68 Linker-Cas fusion, lane 7=holo (+sgRNA) with TniQ-68 Linker-Cas fusion, lane 8=holo (+sgRNA) with TniQ-72 Linker-Cas fusion. FIG. 21D depicts a gel image of PCR 5 (detecting the LE junction to the donor) of transposition: lane 1=apo (no sgRNA) with TniQ-Cas fusion, lane 2=holo (+sgRNA) with TniQ-Cas fusion, lane 3=holo Cas alone, lane 4=apo (no sgRNA) with TniQ-48 Linker-Cas fusion, lane 5=holo (+sgRNA) with TniQ-48 Linker-Cas fusion, lane 6=apo (no sgRNA) with TniQ-68 Linker-Cas fusion, lane 7=holo (+sgRNA) with TniQ-68 Linker-Cas fusion, lane 8=holo (+sgRNA) with TniQ-72 Linker-Cas fusion. FIG. 21E depicts a gel image of PCR 4 (detecting the RE junction to the donor) of transposition: lane 1=apo (no sgRNA), lane 2=holo (+sgRNA), lane 3=apo (no sgRNA) with NLS-TniQ-Cas-NLS fusion, lane 4=holo (+sgRNA) with NLS-TniQ-Cas-NLS fusion, lane 5=apo (no sgRNA) with NLS-TniQ-77 Linker-Cas-NLS fusion, lane 6=holo (+sgRNA) with NLS-TniQ-77 Linker-Cas-NLS fusion. FIG. 21F depicts a gel image of PCR 5 (detecting the LE junction to the donor) of transposition: lane 1=apo (no sgRNA), lane 2=holo (+sgRNA), lane 3=apo (no sgRNA) with NLS-TniQ-Cas-NLS fusion, lane 4=holo (+sgRNA) with NLS-TniQ-Cas-NLS fusion, lane 5=apo (no sgRNA) with NLS-TniQ-77 Linker-Cas-NLS fusion, lane 6=holo (+sgRNA) with NLS-TniQ-77 Linker-Cas-NLS fusion. FIG. 21G depicts a gel image of PCR 4 (detecting the RE junction to the donor) of transposition: lane 1=apo (no sgRNA), lane 2=holo (+sgRNA), lane 3=NLS-TniQ-Cas-NLS apo (no sgRNA), lane 4=NLS-TniQ-Cas-NLS holo (+sgRNA), lane 5=Cas-NLS-P2A-NLS-TniQ apo (no sgRNA), lane 6=Cas-NLS-P2A-NLS-TniQ holo (+sgRNA). FIG. 21H depicts a gel image of PCR 5 (detecting the LE junction to the donor) of transposition: lane 1=apo (no sgRNA), lane 2=holo (+sgRNA), lane 3=NLS-TniQ-Cas-NLS apo (no sgRNA), lane 4=NLS-TniQ-Cas-NLS holo (+sgRNA), lane 5=Cas-NLS-P2A-NLS-TniQ apo (no sgRNA), lane 6=Cas-NLS-P2A-NLS-TniQ holo (+sgRNA).

FIGS. 22A-22F depict the results of expression of TnsB and TnsC in human cells, followed by cell fractionation and in vitro transposition reactions. FIG. 22A depicts a gel image of PCR 4 (detecting the RE junction to the donor) of transposition: lane 1=apo (no sgRNA), lane 2=holo (+sgRNA), lane 3=holo (+sgRNA) with Untreated (no TnsB) cytoplasm, lane 4=holo (+sgRNA) with untreated nucleoplasm, lane 5=holo (+sgRNA) with NLS-TnsB cell cytoplasm, lane 6=holo (+sgRNA) with NLS-TnsB cell nucleoplasm, lane 7=holo (+sgRNA) with TnsB-NLS cell cytoplasm, lane 8=holo (+sgRNA) with TnsB-NLS cell nucleoplasm, lane 9=holo (+sgRNA) with NLS-TniQ cell cytoplasm, lane 10=holo (+sgRNA) with NLS-TniQ cell nucleoplasm. FIG. 22B depicts a gel image of PCR 5 (detecting the LE junction to the donor) of transposition: lane 1=apo (no sgRNA), lane 2=holo (+sgRNA), lane 3=holo (+sgRNA) with Untreated (no TnsB) cytoplasm, lane 4=holo (+sgRNA) with untreated nucleoplasm, lane 5=holo (+sgRNA) with NLS-TnsB cell cytoplasm, lane 6=holo (+sgRNA) with NLS-TnsB cell nucleoplasm, lane 7=holo (+sgRNA) with TnsB-NLS cell cytoplasm, lane 8=holo (+sgRNA) with TnsB-NLS cell nucleoplasm, lane 9=holo (+sgRNA) with NLS-TniQ cell cytoplasm, lane 10=holo (+sgRNA) with NLS-TniQ cell nucleoplasm. FIG. 22C depicts a gel image of PCR 4 (detecting the RE junction to the donor) of transposition: lane 1=apo (no sgRNA), lane 2=holo (+sgRNA), lane 3=holo (+sgRNA) without TnsC, lane 4=holo (+sgRNA) with Untreated (no TnsC) cytoplasm, lane 5=holo (+sgRNA) with untreated nucleoplasm, lane 6=holo (+sgRNA) with NLS-HA-TnsC cell cytoplasm, lane 7=holo (+sgRNA) with NLS-HA-TnsC cell nucleoplasm, lane 8=holo (+sgRNA) with TnsC-NLS cell cytoplasm, lane 9=holo (+sgRNA) with TnsC-NLS cell nucleoplasm. FIG. 22D depicts a gel image of PCR 5 (detecting the LE junction to the donor) of transposition: lane 1=apo (no sgRNA), lane 2=holo (+sgRNA), lane 3=holo (+sgRNA) without TnsC, lane 4=holo (+sgRNA) with Untreated (no TnsC) cytoplasm, lane 5=holo (+sgRNA) with untreated nucleoplasm, lane 6=holo (+sgRNA) with NLS-HA-TnsC cell cytoplasm, lane 7=holo (+sgRNA) with NLS-HA-TnsC cell nucleoplasm, lane 8=holo (+sgRNA) with TnsC-NLS cell cytoplasm, lane 9=holo (+sgRNA) with TnsC-NLS cell nucleoplasm. FIG. 22E depicts a gel image of PCR 4 (detecting the RE junction to the donor) of transposition: lane 1=apo (no sgRNA), lane 2=holo (+sgRNA), lane 3=apo (no sgRNA) NLS-TnsB-IRES-NLS-TnsC cytoplasm, lane 4=holo (+sgRNA) NLS-TnsB-IRES-NLS-TnsC cytoplasm, lane 5=apo (no sgRNA) NLS-TnsB-IRES-NLS-TnsC nucleoplasm, lane 6=holo (+sgRNA) NLS-TnsB-IRES-NLS-TnsC nucleoplasm, lane 7=apo (no sgRNA) TnsB-NLS-IRES-NLS-TnsC cytoplasm, lane 8=holo (+sgRNA) TnsB-NLS-IRES-NLS-TnsC cytoplasm, lane 9=apo (no sgRNA) TnsB-NLS-IRES-NLS-TnsC nucleoplasm, lane 10=holo (+sgRNA) TnsB-NLS-IRES-NLS-TnsC nucleoplasm. FIG. 22F depicts a gel image of PCR 5 (detecting the LE junction to the donor) of transposition: lane 1=apo (no sgRNA), lane 2=holo (+sgRNA), lane 3=apo (no sgRNA) NLS-TnsB-IRES-NLS-TnsC cytoplasm, lane 4=holo (+sgRNA) NLS-TnsB-IRES-NLS-TnsC cytoplasm, lane 5=apo (no sgRNA) NLS-TnsB-IRES-NLS-TnsC nucleoplasm, lane 6=holo (+sgRNA) NLS-TnsB-IRES-NLS-TnsC nucleoplasm, lane 7=apo (no sgRNA) TnsB-NLS-IRES-NLS-TnsC cytoplasm, lane 8=holo (+sgRNA) TnsB-NLS-IRES-NLS-TnsC cytoplasm, lane 9=apo (no sgRNA) TnsB-NLS-IRES-NLS-TnsC nucleoplasm, lane 10=holo (+sgRNA) TnsB-NLS-IRES-NLS-TnsC nucleoplasm.

FIGS. 23A-23G depict the results of expression of Cas12k and TniQ linked constructs in human cells, followed by in vitro transposition testing. FIG. 23A depicts a gel image of PCR 5 (detecting the LE junction to the donor) of transposition: lane 1=apo (no sgRNA), lane 2=holo (+sgRNA), lane 3=Cas-NLS holo (+sgRNA) cytoplasm, lane 4=Cas-NLS holo (+sgRNA) nucleoplasm, lane 5=Cas-NLS holo (+sgRNA) nucleoplasm+additional sgRNA, lane 6=Cas-NLS-P2A-NLS-TniQ holo (+sgRNA) cytoplasm, lane 7=Cas-NLS-P2A-NLS-TniQ holo (+sgRNA) nucleoplasm, lane 8=Cas-NLS-P2A-NLS-TniQ holo (+sgRNA) nucleoplasm+additional sgRNA. FIG. 23B depicts a gel image of PCR 4 (detecting the RE junction to the donor) of transposition: lane 1=apo (no sgRNA), lane 2=holo (+sgRNA), lane 3=apo (no sgRNA) Cas-NLS-P2A-NLS-TniQ cytoplasm, lane 4=holo (+sgRNA) Cas-NLS-P2A-NLS-TniQ cytoplasm, lane 5=apo (no sgRNA) Cas-NLS-P2A-NLS-TniQ nucleoplasm, lane 6=holo (+sgRNA) Cas-NLS-P2A-NLS-TniQ nucleoplasm, lane 7=holo (+sgRNA) Cas-NLS-P2A-NLS-TniQ nucleoplasm+additional holo Cas-NLS, lane 8=holo (+sgRNA) Cas-NLS-P2A-NLS-TniQ nucleoplasm+NLS-TniQ. FIG. 23C depicts a gel image of PCR 5 (detecting the LE junction to the donor) of transposition: lane 1=apo (no sgRNA), lane 2=holo (+sgRNA), lane 3=apo (no sgRNA) Cas-NLS-P2A-NLS-TniQ cytoplasm, lane 4=holo (+sgRNA) Cas-NLS-P2A-NLS-TniQ cytoplasm, lane 5=apo (no sgRNA) Cas-NLS-P2A-NLS-TniQ nucleoplasm, lane 6=holo (+sgRNA) Cas-NLS-P2A-NLS-TniQ nucleoplasm, lane 7=holo (+sgRNA) Cas-NLS-P2A-NLS-TniQ nucleoplasm+additional holo Cas-NLS, lane 8=holo (+sgRNA) Cas-NLS-P2A-NLS-TniQ nucleoplasm+NLS-TniQ. FIG. 23D depicts a gel image of PCR 4 (detecting the RE junction to the donor) of transposition: lane 1=apo (no sgRNA), lane 2=holo (+sgRNA), lane 3=apo (no sgRNA) NLS-TniQ-Cas-NLS cytoplasm, lane 4=holo (+sgRNA) NLS-TniQ-Cas-NLS cytoplasm, lane 5=apo (no sgRNA) NLS-TniQ-Cas-NLS nucleoplasm, lane 6=holo (+sgRNA) NLS-TniQ-Cas-NLS nucleoplasm, lane 7=holo (+sgRNA) NLS-TniQ-Cas-NLS nucleoplasm+additional holo Cas-NLS, lane 8=holo (+sgRNA) NLS-TniQ-Cas-NLS nucleoplasm+NLS-TniQ. FIG. 23E depicts a gel image of PCR 5 (detecting the LE junction to the donor) of transposition: lane 1=apo (no sgRNA), lane 2=holo (+sgRNA), lane 3=apo (no sgRNA) NLS-TniQ-Cas-NLS cytoplasm, lane 4=holo (+sgRNA) NLS-TniQ-Cas-NLS cytoplasm, lane 5=apo (no sgRNA) NLS-TniQ-Cas-NLS nucleoplasm, lane 6=holo (+sgRNA) NLS-TniQ-Cas-NLS nucleoplasm, lane 7=holo (+sgRNA) NLS-TniQ-Cas-NLS nucleoplasm+additional holo Cas-NLS, lane 8=holo (+sgRNA) NLS-TniQ-Cas-NLS nucleoplasm+NLS-TniQ. FIG. 23F depicts a gel image of PCR 4 (detecting the RE junction to the donor) of transposition: lane 1=apo (no sgRNA), lane 2=holo (+sgRNA), lane 3=apo (no sgRNA) Cas-NLS-IRES-NLS-TniQ cytoplasm, lane 4=holo (+sgRNA) Cas-NLS-IRES-NLS-TniQ cytoplasm, lane 5=apo (no sgRNA) Cas-NLS-IRES-NLS-TniQ nucleoplasm, lane 6=apo (no sgRNA) Cas-NLS-IRES-NLS-TniQ nucleoplasm+additional PURExpress, lane 7=apo (no sgRNA) Cas-NLS-IRES-NLS-TniQ nucleoplasm+additional Cas-NLS, lane 8=apo (no sgRNA) Cas-NLS-IRES-NLS-TniQ nucleoplasm+NLS-TniQ, lane 9=holo (+sgRNA) Cas-NLS-IRES-NLS-TniQ nucleoplasm, lane 10=holo (+sgRNA) Cas-NLS-IRES-NLS-TniQ nucleoplasm+additional PURExpress, lane 11=holo (+sgRNA) Cas-NLS-IRES-NLS-TniQ nucleoplasm+additional Cas-NLS, lane 12=holo (+sgRNA) Cas-NLS-IRES-NLS-TniQ nucleoplasm+NLS-TniQ. FIG. 23G depicts a gel image of PCR 5 (detecting the LE junction to the donor) of transposition: lane 1=apo (no sgRNA), lane 2=holo (+sgRNA), lane 3=apo (no sgRNA) Cas-NLS-IRES-NLS-TniQ cytoplasm, lane 4=holo (+sgRNA) Cas-NLS-IRES-NLS-TniQ cytoplasm, lane 5=apo (no sgRNA) Cas-NLS-IRES-NLS-TniQ nucleoplasm, lane 6=apo (no sgRNA) Cas-NLS-IRES-NLS-TniQ nucleoplasm+additional PURExpress, lane 7=apo (no sgRNA) Cas-NLS-IRES-NLS-TniQ nucleoplasm+additional Cas-NLS, lane 8=apo (no sgRNA) Cas-NLS-IRES-NLS-TniQ nucleoplasm+NLS-TniQ, lane 9=holo (+sgRNA) Cas-NLS-IRES-NLS-TniQ nucleoplasm, lane 10=holo (+sgRNA) Cas-NLS-IRES-NLS-TniQ nucleoplasm+additional PURExpress, lane 11=holo (+sgRNA) Cas-NLS-IRES-NLS-TniQ nucleoplasm+additional Cas-NLS, lane 12=holo (+sgRNA) Cas-NLS-IRES-NLS-TniQ nucleoplasm+NLS-TniQ.

FIG. 24 depicts electrophoretic mobility shift assay (EMSA) results of the 64-1 TnsB and its LE DNA sequence. The EMSA results confirm binding and TnsB recognition. The TnsB protein was expressed in an in vitro transcription/translation system, incubated with FAM-labeled DNA containing the LE sequence, and then separated on a native 5% TBE gel. Binding is observed as a shift upwards in the labeled band. Multiple TnsB binding sites leads to multiple shifts in the EMSA. Lane 1: FAM-labeled DNA only. Lane 2: FAM DNA plus the in vitro transcription/translation system (no TnsB protein). Lane 3: FAM DNA plus TnsB.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

MG64

SEQ ID NOs: 1, 12, 16, 20-30, 64, and 80-85 show the full-length peptide sequences of MG64 Cas effectors.

SEQ ID Nos: 2-4, 13-15, 17-19, and 65-67 show the peptide sequences of MG64 transposition proteins that may comprise a recombinase complex associated with the MG64 Cas effector.

SEQ ID NOs: 5-6, 32-33, 94-95, and 104-105 show nucleotide sequences of MG64 tracrRNAs derived from the same loci as a MG64 Cas effector.

SEQ ID NOs: 7 and 34-35 show nucleotide sequences of MG64 target CRISPR repeats.

SEQ ID NOs: 106-108 show nucleotide sequences of MG64 crRNAs.

SEQ ID NO: 8,10, 39-44, 77, 79, and 93 show nucleotide sequences of right-hand transposase recognition sequences associated with a MG64 system.

SEQ ID NO: 9,11, 36-38, 76, and 78 show nucleotide sequences of left-hand transposase recognition sequences associated with a MG64 system.

SEQ ID NO: 31 shows a PAM sequence associated with MG64 Cas Effectors described herein.

Seq ID NOs: 45-63, 68-75, and 96-103 show nucleotide sequences of single guide RNAs engineered to function with MG64 Cas effectors.

Other Sequences

SEQ ID NOs: 86-87 show peptide sequences of nuclear localizing signals.

SEQ ID NOs: 88-89 show peptide sequences of linkers.

SEQ ID NOs: 90-92 show peptide sequences of epitope tags.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

The term “tracrRNA” or “tracr sequence”, as used herein, can generally refer to a nucleic acid with at least about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes S. aureus, etc. or SEQ ID NOs: *_*). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of ; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

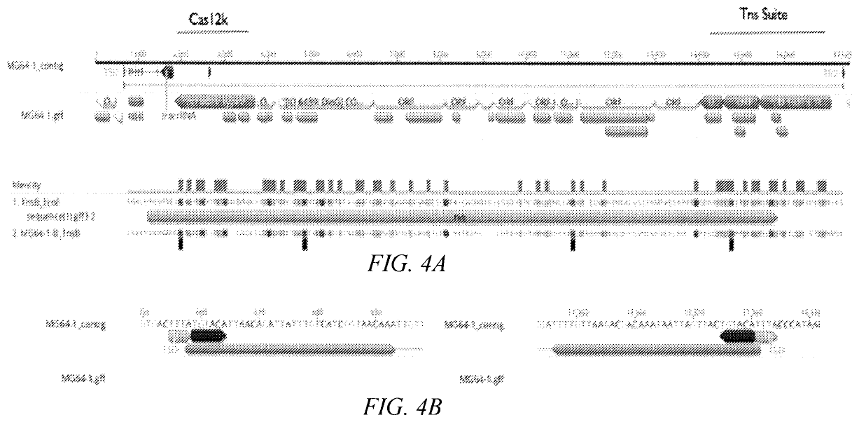

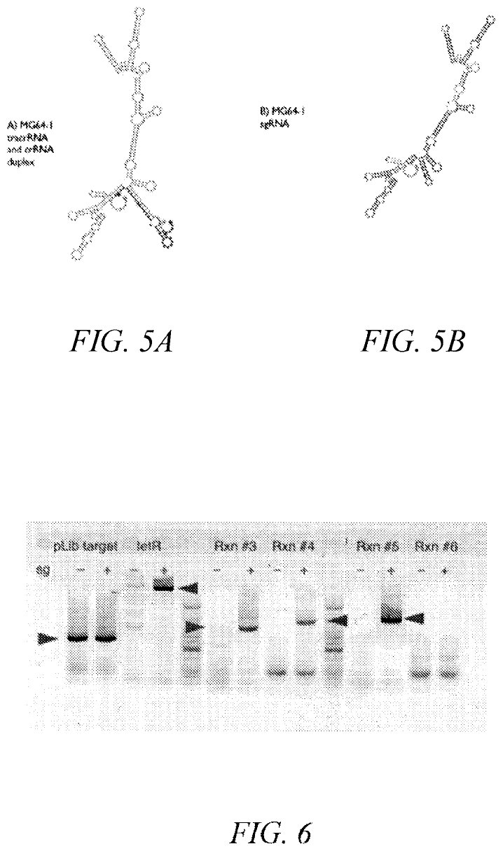

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g. non-conserved residues without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity any one of the systems described herein (e.g., MG64 systems described herein). In some embodiments, such conservatively substituted variants are functional variants. Such functional variants can encompass sequences with substitutions such that the activity of critical active site residues of the endonuclease are not disrupted. In some embodiments, a functional variant of any of the systems described herein lack substitution of at least one of the conserved or functional residues called out in FIG. 4 and FIGS. 5A and 5B. In some embodiments, a functional variant of any of the systems described herein lacks substitution of all of the conserved or functional residues called out in FIG. 4 and FIGS. 5A and 5B.

Conservative substitution tables providing functionally similar amino acids are available from a variety of references (see, for example, Creighton, Proteins: Structures and Molecular Properties (W H Freeman & Co.; 2^{nd} Edition (December 1993))). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

As used herein, the term “RuvC III domain” generally refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to known domain sequences, structural alignment to proteins with annotated domains, or by comparison to Hidden Markov Models (HMMs) built based on known domain sequences (e.g., Pfam HMM PF18541 for RuvC_III).

As used herein, the term “HNH domain” generally refers to an endonuclease domain having characteristic histidine and asparagine residues. An HNH domain can generally be identified by alignment to known domain sequences, structural alignment to proteins with annotated domains, or by comparison to Hidden Markov Models (HMMs) built based on known domain sequences (e.g., Pfam HMM PF01844 for domain HNH).

As used herein, the term “recombinase” generally refers to a site-specific enzyme that mediates the recombination of DNA between recombinase recognition sequences, which results in the excision, integration, inversion, or exchange (e.g., translocation) of DNA fragments between the recombinase recognition sequences.

As used herein, the term “recombine,” or “recombination,” in the context of a nucleic acid modification (e.g., a genomic modification), generally refers to the process by which two or more nucleic acid molecules, or two or more regions of a single nucleic acid molecule, are modified by the action of a recombinase protein. Recombination can result in, inter alia, the insertion, inversion, excision, or translocation of a nucleic acid sequence, e.g., in or between one or more nucleic acid molecules.

As used herein, the term “transposon” generally refers to mobile elements that move in and out of genomes carrying “cargo DNA” with them. In some cases, these transposons may differ on the type of nucleic acid to transpose, the type of repeat at the ends of the transposon, the type of cargo to be carried or by the mode of transposition (i.e. self-repair or host-repair). As used herein, the term “transposase” or “transposases” generally refers to an enzyme that binds to the end of a transposon and catalyzes its movement to another part of the genome. In some cases, the movement may be by a cut and paste mechanism or a replicative transposition mechanism.

As used herein, the term “Tn7” or “Tn7-like transposase” generally refers to a family of transposases comprising three main components: a heteromeric transposase (TnsA and/or TnsB) alongside a regulator protein (TnsC). In addition to the TnsABC transposition proteins, Tn7 elements can encode dedicated target site-selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ. TniQ has been shown to target transposition into resolution sites of plasmids.

In some cases, the CAST systems described herein may comprise one or more Tn7 or Tn7 like transposases. In certain example embodiments, the Tn7 or Tn7 like transposase comprises a multimeric protein complex. In certain example embodiments, the multimeric protein complex comprises TnsA, TnsB, TnsC, or TniQ. In these combinations, the transposases (TnsA, TnsB, TnsC, TniQ) may form complexes or fusion proteins with each other.

As used herein, the term “Cas12k”(alternatively “class II, type V-K”) generally refers to a subtype of Type V CRISPR systems that have been found to be defective in nuclease activity (e.g. they may comprise at least one defective RuvC domain that lacking at least one catalytic residue important for DNA cleavage). Such subtype of effectors have been generally associated with CAST systems.

Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems known and speed the discovery of new oligonucleotide editing functionalities. A recent example of the fruitfulness of such an approach is demonstrated by the 2016 discovery of CasX/CasY CRISPR systems from metagenomic analysis of natural microbial communities.

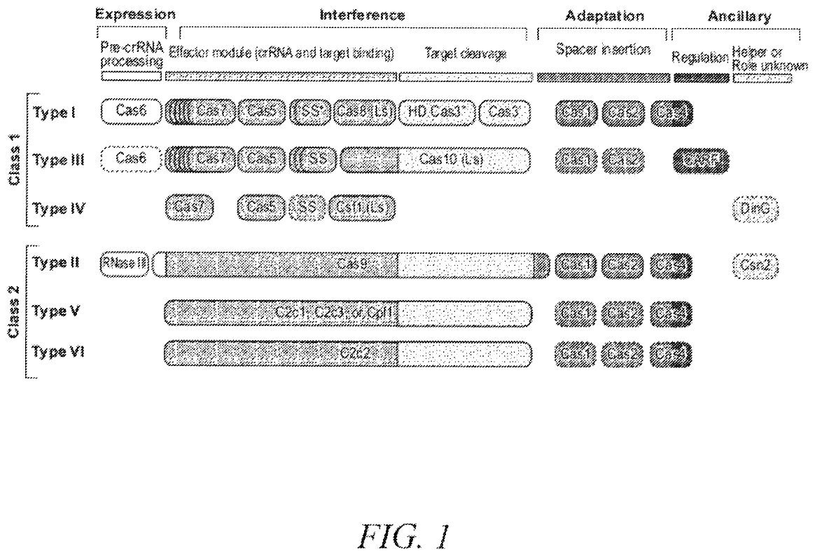

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity (see FIG. 1).

Class I CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that consists of a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type 2 effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRIPSR-Cas systems have RNA-guided RNA endonucleases. Instead of RuvC-like domains, the single polypeptide effector of Type VI systems (e.g. Cas13) comprises two HEPN ribonuclease domains. Differing from both Type II and V systems, Type VI systems also appear to not need a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class II CRISPR-Cas have been most widely adopted for engineering and development as designer nuclease/genome editing applications.

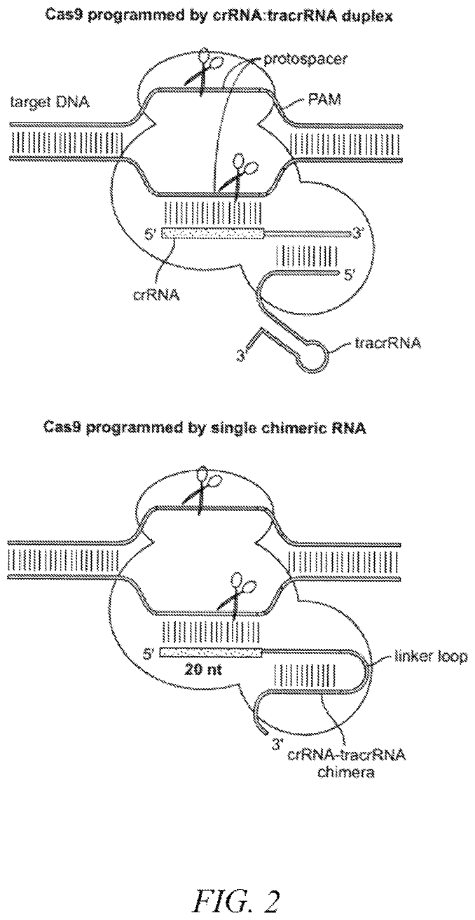

One of the early adaptations of such a system for in vitro use can be found in Jinek et al. (Science. 2012 Aug. 17;337(6096):816-21, which is entirely incorporated herein by reference). The Jinek study first described a system that involved (i) recombinantly-expressed, purified full-length Cas9 (e.g., a Class II, Type II Cas enzyme) isolated from S. pyogenes SF370, (ii) purified mature ˜42 nt crRNA bearing a ˜20 nt 5′ sequence complementary to the target DNA sequence desired to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg²⁺. Jinek later described an improved, engineered system wherein the crRNA of (ii) is joined to the 5′ end of (iii) by a linker (e.g., GAAA) to form a single fused synthetic guide RNA (sgRNA) capable of directing Cas9 to a target by itself (compare top and bottom panel of FIG. 2).

Mali et al. (Science. 2013 Feb. 15; 339(6121): 823-826.), which is entirely incorporated herein by reference, later adapted this system for use in mammalian cells by providing DNA vectors encoding (i) an ORF encoding codon-optimized Cas9 (e.g., a Class II, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

Transposons are mobile elements that can move between positions in a genome. Such transposons have evolved to limit the negative effects they exert on the host. A variety of regulatory mechanisms are used to maintain transposition at a low frequency and sometimes coordinate transposition with various cell processes. Some prokaryotic transposons also can mobilize functions that benefit the host or otherwise help maintain the element. Certain transposons may have also evolved mechanisms of tight control over target site selection, the most notable example being the Tn7 family.

Transposon Tn7 and similar elements may be reservoirs for antibiotic resistance and pathogenesis functions in clinical settings, as well as encoding other adaptive functions in natural environments. The Tn7 system, for example, has evolved mechanisms to almost completely avoid integrating into important host genes, but also maximize dispersal of the element by recognizing mobile plasmids and bacteriophage capable of moving Tn7 between host bacteria.

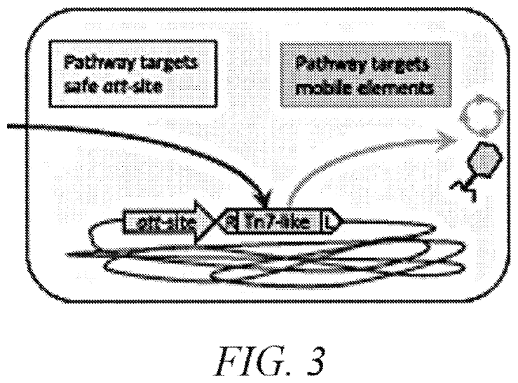

Tn7 and Tn7-like elements may control where and when they insert, possessing one pathway that directs insertion into a single conserved position in bacterial genomes and a second pathway that appears to be adapted to maximizing targeting into mobile plasmids capable of transporting the element between bacteria (see FIG. 3). The association between Tn7-like transposons and CRISPR-Cas systems suggests that the transposons might have hijacked CRISPR effectors to generate R-loops in target sites and facilitate the spread of transposons via plasmids and phages.

MG64 Systems

In one aspect, the present disclosure provides for a system for transposing a cargo nucleotide sequence to a target nucleic acid site. The system may comprise a first double-stranded nucleic acid comprising a cargo nucleotide sequence. This cargo nucleotide sequence may be configured to interact with a Tn7 type transposase complex. The system may comprise a Cas effector complex. The Cas effector complex may comprise a class II, type V Cas effector and an engineered guide polynucleotide configured to hybridize to the target nucleotide sequence. The system may comprise a Tn7 type transposase complex configured to bind the Cas effector complex, wherein the Tn7 type transposase complex comprises a TnsB subunit.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the system further comprises a second double-stranded nucleic acid comprising the target nucleic acid site. In some cases, the system further comprises a PAM sequence compatible with the Cas effector complex adjacent to the target nucleic acid site. In some cases, the PAM sequence is located 3′ of the target nucleic acid site.

In some cases, the engineered guide polynucleotide is configured to bind the class II, type V Cas effector. In some cases, the class II, type V Cas effector is a class II, type V-K effector. In some cases, the class II, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1, 12, 16, 20-30, 64, or 80-85, or a variant thereof. In some cases, the class II, type V Cas effector comprises a polypeptide comprising a sequence substantially identical to SEQ ID NO: 1, 12, 16, 20-30, 64, or 80-85. In some cases, the TnsB subunit comprises a polypeptide having a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 13, 17, or 65, or a variant thereof. In some cases, the TnsB subunit comprises a polypeptide having a sequence substantially identical to SEQ ID NO: 2, 13, 17, or 65.

In some cases, the Tn7 type transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, or 66-67, or a variant thereof. In some cases, the recombinase complex comprises at least one polypeptide comprising a sequence substantially identical to any one of SEQ ID NOs: 3-4, 14-15, 18-19, or 66-67. In some cases, the Tn7 type transposase complex comprises at least two polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, or 66-67, or a variant thereof. In some cases, the Tn7 type transposase complex comprises at least two polypeptides comprising a sequence substantially identical to any one of SEQ ID NOs: 3-4, 14-15, 18-19, or 66-67.

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 5-6, 32-33, 94-95, or 104-105, or a variant thereof. In some cases, the engineered guide polynucleotide comprises a sequence comprising at least about 46-80 consecutive nucleotides substantially identical to any one of SEQ ID NOs: 5-6, 32-33, 94-95 or 104-105.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 9, 11, 36-38, 76, or 78, or a variant thereof. In some cases, the left-hand recombinase sequence comprises a sequence substantially identical to SEQ ID NO: 9, 11, 36-38, 76, or 78.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 8, 10, 39-44, 77, 79, or 93, or a variant thereof. In some cases, the right-hand recombinase sequence comprises a sequence substantially identical to SEQ ID NO: 8, 10, 39-44, 77, 79, or 93.

In some cases, the class II, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

In one aspect, the present disclosure provides for a method for transposing a cargo nucleotide sequence to a target nucleic acid site comprising a target nucleotide sequence comprising expressing a system described herein within a cell or introducing a system described herein to a cell.

In one aspect, the present disclosure provides for a method for transposing a cargo nucleotide sequence to a target nucleic acid site, comprising contacting a first double-stranded nucleic acid comprising the cargo nucleotide sequence with a Cas effector complex comprising a class II, type V Cas effector and at least one engineered guide polynucleotide configured to hybridize to the target nucleotide sequence. The method may comprise contacting the first double-stranded nucleic acid comprising the cargo nucleotide sequence with a Tn7 type transposase complex configured to bind the Cas effector complex, wherein the Tn7 type transposase complex comprises a TnsB subunit. The method may comprise contacting the first double-stranded nucleic acid comprising the cargo nucleotide sequence with a second double-stranded nucleic acid comprising the target nucleic acid site.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the method further comprises a PAM sequence compatible with the Cas effector complex adjacent to the target nucleic acid site. In some cases, the PAM sequence is located 3′ of the target nucleic acid site.

In some cases, the engineered guide polynucleotide is configured to bind the class II, type V Cas effector. In some cases, the class II, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1, 12, 16, 20-30, 64, or 80-85, or a variant thereof. In some cases, the class II, type V Cas effector comprises a polypeptide comprising a sequence substantially identical to SEQ ID NO: 1, 12, 16, 20-30, 64, or 80-85.

In some cases, the TnsB subunit comprises a polypeptide having a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 13, 17, or 65, or a variant thereof. In some cases, the TnsA subunit comprises a polypeptide having a sequence substantially identical to SEQ ID NO: 2, 13, 17, or 65.

In some cases, the Tn7 type transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, or 66-67, or a variant thereof. In some cases, the recombinase complex comprises at least one polypeptide comprising a sequence substantially identical to any one of SEQ ID NOs: 3-4, 14-15, 18-19, or 66-67. In some cases, the Tn7 type transposase complex comprises at least two polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, or 66-67, or a variant thereof. In some cases, the Tn7 type transposase complex comprises at least two polypeptides comprising a sequence substantially identical to any one of SEQ ID NOs: 3-4, 14-15, 18-19, or 66-67.

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 5-6, 32-33, 94-95, or 104-105, or a variant thereof. In some cases, the engineered guide polynucleotide comprises a sequence comprising at least about 46-80 consecutive nucleotides substantially identical to any one of SEQ ID NOs: 5-6, 32-33, 94-95 or 104-105.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 9, 11, 36-38, 76, or 78, or a variant thereof. In some cases, the left-hand recombinase sequence comprises a sequence substantially identical SEQ ID NO: 9, 11, 36-38, 76, or 78. In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 8, 10, 39-44, 77, 79, or 93, or a variant thereof. In some cases, the right-hand recombinase sequence comprises a sequence substantially identical to SEQ ID NO: 8, 10, 39-44, 77, 79, or 93.

In some cases, the class II, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G

### EXAMPLES

### Example 1—(General Protocol) PAM Sequence Identification/Confirmation for Systems Described Herein

Putative endonucleases were expressed in an E. coli lysate-based expression system (myTXTL, Arbor Biosciences). PAM sequences were determined by sequencing plasmids containing randomly-generated potential PAM sequences that could be cleaved by the putative nucleases. In this system, an E. coli codon optimized nucleotide sequence encoding the putative nuclease was transcribed and translated in vitro from a PCR fragment under control of a T7 promoter. A second PCR fragment with a minimal CRISPR array composed of a T7 promoter followed by a repeat-spacer-repeat sequence was transcribed in the same reaction. Successful expression of the endonuclease and repeat-spacer-repeat sequence in the TXTL system followed by CRISPR array processing provided active in vitro CRISPR nuclease complexes.

A library of target plasmids containing a spacer sequence matching that in the minimal array preceded by 8N mixed bases (potential PAM sequences) was incubated with the output of the TXTL reaction. After 1-3 hr, the reaction was stopped and the DNA was recovered via a DNA clean-up kit, e.g., Zymo DCC, AMPure XP beads, QiaQuick etc. Adapter sequences were blunt-end ligated to DNA with active PAM sequences that were cleaved by the endonuclease, whereas DNA that was not cleaved was inaccessible for ligation. DNA segments comprising active PAM sequences were then amplified by PCR with primers specific to the library and the adapter sequence. The PCR amplification products were resolved on a gel to identify amplicons that correspond to cleavage events. The amplified segments of the cleavage reaction were also used as templates for preparation of an NGS library or as a substrate for Sanger sequencing. Sequencing this resulting library, which is a subset of the starting 8N library, revealed sequences with PAM activity compatible with the CRISPR complex. For PAM testing with a processed RNA construct, the same procedure was repeated except that an in vitro transcribed RNA was added along with the plasmid library and the minimal CRISPR array template was omitted.

Analysis of the intergenic regions surrounding the Cas effector and CRISPR array identified a potential anti-repeat sequence corresponding to the duplexing sequence of the tracrRNA. TracrRNA and crRNA repeat were folded and trimmed, adding a tetraloop sequence of GAAA to maintain the stem loop region of the crRNA-tracrRNA complex.

### Example 2a—In vitro Targeted Integrase Activity

Integrase activity was preferentially assayed with a previously identified PAM but may be conducted with a PAM library substrate instead, with reduced efficiency. One arrangement of components for in vitro testing involved three plasmids other than that containing the donor sequence: (1) an expression plasmid with effector (or effectors) under a T7 promoter; (2) an expression plasmid with transposase genes under a T7 promoter; a sgRNA or crRNA and tracrRNA; (3) a target plasmid which contained the spacer site and appropriate PAM; and (4) a donor plasmid which contained the required left end (LE) and right end (RE) DNA sequences for transposition around a cargo gene (e.g. a selection marker such as a Tet resistance gene). Using an in vitro transcription/translation (TXTL) system (e.g. E. coli lysate- or reticulocyte lysate-based system), the effector and transposase genes were expressed. After expression, the RNA, target DNA, and donor DNA were added and incubated to allow for transposition to occur. Transposition was detected via PCR across the junction of the transposase site, with one primer on the target DNA and one primer on the donor DNA. The resulting PCR product was sequenced via NGS to determine the exact insertion topology relative to the sgRNA/crRNA targeted site. The primers were located downstream such that a variety of insertion sites were accommodated and detected. Primers were designed such that integration was detected in either orientation of cargo and on either side of the spacer, as the integration direction was also not known initially.

Integration efficiency was measured via quantitative PCR (qPCR) measurements of the experimental output of target DNA with integrated cargo, normalized to the amount of unmodified target DNA also measured via qPCR.

This assay may be conducted with purified protein components rather than from lysate-based expression. In this case the proteins were expressed in an E. coli protease deficient B strain under a T7 inducible promoter, the cells were lysed using sonication, and the His-tagged protein of interest was purified using HisTrap FF (GE Lifescience) Ni-NTA affinity chromatography on the AKTA Avant FPLC (GE Lifescience). Purity was determined using densitometry in ImageLab software (Bio-Rad) of the protein bands resolved on SDS-PAGE and InstantBlue Ultrafast (Sigma-Aldrich) Coomassie stained acrylamide gels (Bio-Rad). The protein was desalted in storage buffer composed of 50 mM Tris-HCl, 300 mM NaCl, 1 mM TCEP, 5% glycerol; pH 7.5 (or other buffers as determined for maximum stability) and stored at −80° C. After purification the effector(s) and transposase(s) were added to the sgRNA, target DNA, and donor DNA as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl₂, 28 mM NaCl, 21 mM KCl, 1.35% glycerol,(final pH 7.5) supplemented with 15 mM Mg(Oac)₂.

### Example 2b—In vitro Activity

Targeted Nuclease

In situ expression and protein sequence analyses indicated that some RNA guided effectors are active nucleases. They contained predicted endonuclease-associated domains (matching RuvC and HNH_endonuclease domains), and/or predicted HNH and RuvC catalytic residues.

Candidate activity was tested with engineered single guide RNA sequences using the myTXTL system and in vitro transcribed RNA. Active proteins that successfully cleaved the library yielded a band around 170 bp in the gel.

DNA Integration and Transposition

Transposons are predicted to be active when the genomic sequences encoding them contain one or more protein sequences with transposase and/or integrase function within the left and right ends of the transposon. A Tn7 transposon, as defined here, consists of a catalytic transposase TnsB, but may also contain TnsA, TnsC, TnsD, TnsE, TniQ, and/or other transposase or integrases. The transposon ends consist of predicted transposase binding sites, which contain direct and/or inverted repeats of 15 bp to 150 bp in length flanking the transposase proteins and other ‘cargo’ genes. Protein sequence analysis indicated that the transposases contain integrase domains, transposase domains and/or transposase catalytic residues, suggesting that they are active (e.g. FIG. 4A).

Targeted DNA Integration

Putative CRISPR-associated transposons (CAST) contain a DNA and/or RNA targeting CRISPR nuclease or effector and proteins with predicted transposase function in the vicinity of a CRISPR array. In some systems, the nuclease is predicted to be active based on the presence of endonuclease-associated catalytic domains and/or catalytic residues.

In some systems, the effector is predicted to have homology with known CRISPR effector proteins, but to be inactive based on the absence of endonuclease domains and/or catalytic residues. The transposases are predicted to be associated with the effector when the CRISPR loci (inactive CRISPR nuclease and array) and the transposase proteins are located within the predicted transposon left and right ends (FIG. 4A). In this case, the effector is predicted to direct DNA integration to specific genomic locations based on a guide RNA.

CAST activity was tested with five types of components (1) a Cas effector protein expressed by myTXTL or PURExpress, (2) a target DNA fragment or plasmid containing the target sequence and PAM corresponding to the Cas enzyme, (3) a donor DNA fragments containing a marker or fragment of DNA flanked by the LE and RE of the transposase system in a DNA fragment or plasmid (4) any combination of transposase proteins expressed using myTXTL or PURExpress, and (5) an engineered in vitro transcribed single guide RNA sequence. Active systems that successfully transposed the donor fragment were assayed by PCR amplification of the donor-target junction.

After performing the transposition reaction, PCR amplification of the junction showed that proper donor-target formation was made, and the transposition reaction was sg dependent. (FIG. 6). PCR amplification of reactions #3 and #4 indicated that both orientations of the donor relative to the target were made: one where the LE is closer to the PAM, and one where the RE is closer to the PAM. While both transposition orientations were made, there was a preference for donor integration in the target where the LE is closer to the PAM, represented by strong band present for reactions #4 and #5.

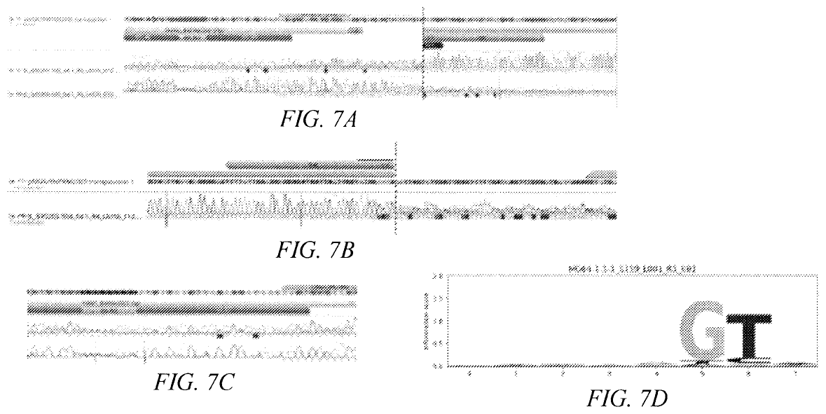

Sanger sequencing of the preferred orientation product was performed. Of the integrations that occurred with the LE closer to the PAM, there was a clear degradation of the sequencing chromatogram signal from either the forward or reverse direction over the target/donor junction. This indicated that, of the products that were oriented with the LE closer to the PAM, integration occurred in a range of nucleotides, with the primary product of LE-closer-to-PAM products as a 61 bp integration from the PAM (FIG. 7A). Sequencing that originated from the donor over the donor-target junction defined the composition of the essential outer bounds of the LE and RE sequences (FIGS. 7A and 7B). Further investigation of the LE and RE domains will determine the inner limits of the LE and RE sequences that are essential for transposition. Sequencing of the RE on LE-closer-to-PAM products showed a 3 bp duplication downstream of the donor RE (FIG. 7B). This is in part due to the Tn7 transposase integration event that cleaved and ligated the donor fragment at a staggered cut site. A 3 bp duplication is smaller than the expected 5 bp of duplication from other Tn7 transposases.

Sanger sequencing of the PCR amplified product over the 8N library of the target plasmid also elucidated that the PAM preference of the MG64-1 effector as a nGTn/nGTt on the 5′ end of the spacer (FIG. 7C). NGS analysis of the PAM library target corroborated the nGTn motif preference at the 5′ end.

### Example 3—Predicted RNA Folding

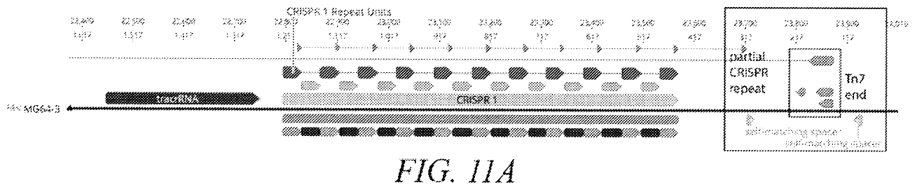

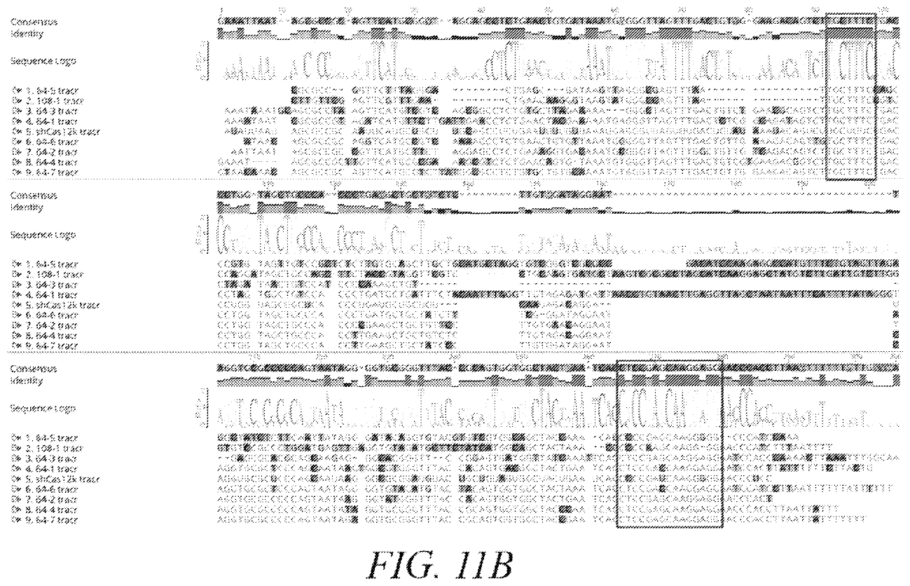

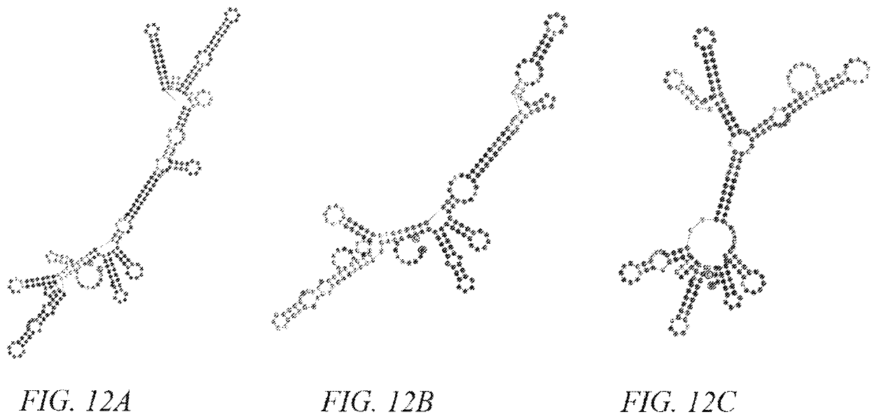

Predicted RNA folding of the active single RNA sequence was computed at 3T using the method of Andronescu 2007. All hairpin-loop secondary structures were singly deleted from the structure and iteratively compiled into a smaller single guide. In a second approach, the tracrRNA of MG64-1 was aligned to known type Vk tracrRNA, and areas of unique insertions were mutated out of the single guide, and minimized by 57 bases. FIG. 12A depicts the predicted structure of MG64-1 sgRNA. FIG. 12B depicts the predicted structure of MG64-3 sgRNA. FIG. 12C depicts the predicted structure of MG64-5 sgRNA. The color of the bases corresponds to the probability of base pairing of that base, wherein red represents high probability and blue represents low probability.

### Example 4—Transposon End Verification Via Gel Shift

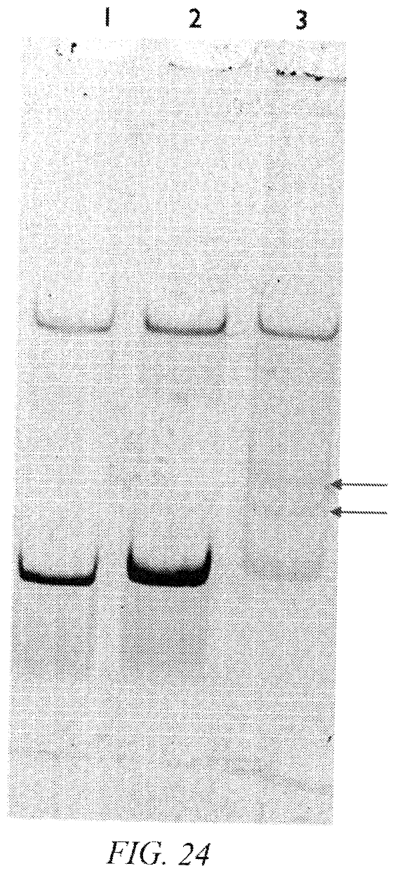

The transposon ends were tested for TnsB binding via an electrophoretic mobility shift assay (EMSA). In this case the potential LE or RE was synthesized as a DNA fragment (100-500 bp) and end-labeled with FAM via PCR with FAM-labeled primers. The TnsB protein was synthesized in an in vitro transcription/translation system (e.g. PURExpress). After synthesis, 1 μL, of TnsB protein was added to 50 nM of the labeled RE or LE in a 10 μL, reaction in binding buffer (20 mM HEPES pH 7.5, 2.5 mM Tris pH 7.5, 10 mM NaCl, 0.0625 mM EDTA, 5 mM TCEP, 0.005% BSA, 1 ug/mL poly(dI-dC), and 5% glycerol). The binding was incubated at 30° for 40 minutes, then 2 uL of 6X loading buffer (60 mM KCl, 10 mM Tris pH 7,6, 50% glycerol) was added. The binding reaction was separated on a 5% TBE gel and visualized. Shifts of the LE or RE in the presence of TnsB were attributed to successful binding and were indicative of transposase activity (FIG. 24).

### Example 5—Integrase Activity in E. coli

As E. coli lacks the capacity to efficiently repair genomic double-stranded DNA breaks, transformation of E. coli by agents able to cause double-stranded breaks in the E. coli genome causes cell death. Exploiting this phenomenon, endonuclease or effector-assisted integrase activity was tested in E. coli by recombinantly expressing either the endonuclease or effector-assisted integrase and a guide RNA (determined e.g. as in Example 3) in a target strain with spacer/target and PAM sequences integrated into its genomic DNA.

Engineered strains were then transformed with a plasmid containing the nuclease or effector with single guide RNA, a plasmid expressing the integrase and accessory genes, and a plasmid containing a temperature sensitive origin of replication with a selectable marker flanked by left end (LE) and right end (RE) transposon motifs for integration. Transformants induced for expression of these genes were then screened for transfer of the marker to the genomic target by selection at restrictive temperature for plasmid replication and the marker integration in the genome was confirmed by PCR.

Off target integrations were screened using an unbiased approach. In brief, purified gDNA was fragmented with Tn5 transposase or shearing, and DNA of interest was then PCR amplified using primers specific to a ligated adaptor and the selectable marker. The amplicons were then prepared for NGS sequencing. Analysis of the resulting sequences were trimmed of the transposon sequences and flanking sequences were mapped to the genome to determine insertion position, and off target insertion rates were determined.

### Example 6—Colony PCR Screen of Transposase Activity

For testing of nuclease or effector assisted integrase activity in bacterial cells, strain MGB0032 was constructed from BL21(DE3) E. coli cells which were engineered to contain the target and corresponding PAM sequence specific to MG64_1. MGB0032 E. coli cells were then transformed with pJL56 (plasmid that expresses the MG64_1 effector and helper suite, ampicillin resistant) and pTCM 64_1 sg, a chloramphenicol-resistant plasmid that expresses the single guide RNA sequence for the engineered target of interest driven by a T7 promoter.

An MGB0032 culture containing both plasmids was then grown to a saturation, diluted at least 1:10 into growth culture with appropriate antibiotics, and incubated at 37° C. until OD of approximately 1. Cells from this growth stage were made electrocompetent and transformed with streamlined 64_1 pDonor, a plasmid bearing a tetracycline resistance marker flanked by left end (LE) and right end (RE) transposon motifs for integration. Electroporated cells were then recovered for 2 hours on LB medium in the presence or absence of IPTG at a final concentration of 100 μM before being plated on LB-agar-ampicillin-chloramphenicol-tetracycline and incubated 4 days at 37° C. Sterile toothpicks were used to sample each resultant CFU, which was mixed into water. To this solution was added Q5 High Fidelity PCR mastermix (New England Biolabs) and primers LA155 (5′-GCTCTTCCGATCTNNNNNGATGAGCGCATTGTTAGATTTCAT-3′) and oJL50 (5′-AAACCGACATCGCAGGCTTC-3′). These primers flank the predicted insertion junction. The predicted product size was 609 bp. DNA amplified PCR product was visualized on a 2% agarose gel. Sanger sequencing of PCR products confirmed the transposition event.

### Example 7—In Cell Expression/in Vitro Assay

To test the functionality of the NLS constructs in a physiologically relevant environment, constructs cloned with active NLS-tagged CAST components were integrated into K562 cells using lentiviral transduction. Briefly, constructs cloned into lentiviral transfer plasmids were transfected into 293T cells with envelope and packaging plasmids, and virus containing supernatant was harvested from the media after 72 hr incubation. Media containing virus was then incubated with K562 cell lines with 8 μg/mL of polybrene for 72 hrs, and transfected cells were then selected for integration in bulk using Puromycin at 1 μg/mL for 4 days. Cell lines undergoing selection were harvested at the end of 4 days, and differentially lysed for nuclear and cytoplasmic fractions. Subsequent fractions were then tested for transposition capability with a complementary set of in vitro expressed components.

10 million cells were centrifuged and washed once with 1xPBS pH7.4. Supernatant wash was aspirated completely to the cell pellet, and flash frozen at −80C for 16 hrs. After thawing on ice, cell pellet size was measured by mass, and appropriate extraction volumes of cell fractionation and nuclear extraction reagent (NE-PER) was used to natively extract proteins in cell fractions. Briefly, cytoplasmic extraction reagent was used at 1:10 mass of cells to volume of extraction reagent. Cell suspension was mixed by vortexing and lysed with non-ionic detergent. Cells were then centrifuged at 16,000×g at 4° C. for 5 minutes. Cytoplasmic extraction supernatant was then decanted and saved for in vitro testing. Nuclear extraction reagent was then added 1:2 original cell mass to nuclear extraction reagent and incubated on ice for 1 hr on ice with intermittent vortexing. Nuclear suspension was then centrifuged at 16,000×g for 10 minutes at 4° C. and supernatant nuclear extract was decanted and tested for in vitro transposition activity. Using 4 μL of each cell and nuclear extract for each condition, we performed the in vitro transposition reaction with a complementary set of in vitro expressed proteins, donor DNA, pTarget, and buffer. Evidence of transposition activity was assayed by PCR amplification of donor-target junctions.

### Example 8—Activity in Mammalian Cells (Prophetic)

To show targeting and cleavage activity in mammalian cells, nuclear localization sequences are fused to the C terminus of each of the nuclease or effector proteins and integrase proteins and the fusion proteins are purified. A single guide RNA targeting a genomic locus of interest is synthesized and incubated with the nuclease/effector protein to form a ribonucleoprotein complex. Cells are transfected with a plasmid containing a selectable neomycin resistance marker (NeoR) or a fluorescent marker flanked by the left end (LE) and right end (RE) motifs, recovered for 4-6 hours, and subsequently electroporated with nuclease RNP and integrase proteins. Integration of a plasmid into the genome is quantified by counting G418-resistant colonies or fluorescence activated cell cytometry. Genomic DNA is extracted 72 hours after electroporation and used for the preparation of an NGS-library. Off target frequency is assayed by fragmenting the genome and preparing amplicons of the transposon marker and flanking DNA for NGS library preparation. At least 40 different target sites are chosen for testing each targeting system's activity.

### Example 9—Activity of Targeted Nuclease

In situ expression and protein sequence analyses suggested that some RNA guided effectors are active nucleases. They contain predicted endonuclease-associated domains (matching RuvC and HNH_endonuclease domains) and predicted HNH and RuvC catalytic residues (FIG. 4A).

Candidate activity was tested with engineered single guide RNA sequences using the myTXTL system and in vitro transcribed RNA. Active proteins that successfully cleaved the library yielded a band around 170 bp in the gel.

### Example 10—Identification of Transposons

Transposons are predicted to be active when they contain one or more protein sequences with transposase and/or integrase function between the left and right ends of the transposon. A Tn7 transposon, as defined here, consists of a catalytic transposase TnsB, but may also contain TnsA, TnsC, TnsD, TnsE, TniQ, and/or other transposases or integrases. The transposon ends consist of predicted transposase binding sites, which contain direct and/or inverted repeats of 15 bp to 150 bp in length flanking the transposase proteins and other ‘cargo’ genes. Protein sequence analysis indicated that the transposases contain integrase domains, transposase domains and/or transposase catalytic residues, suggesting that they are active (e.g. FIG. 4A and FIG. 5A).

### Example 11—Identification of CRISPR-Associated Transposons

Putative CRISPR-associated transposons (CAST) contain a DNA and/or RNA targeting CRISPR effector and proteins with predicted transposase function in the vicinity of a CRISPR array. In some systems, the effector is predicted to have nuclease activity based on the presence of endonuclease-associated catalytic domains and/or catalytic residues (e.g. FIG. 4A). The transposases were predicted to be associated with the active nucleases when the CRISPR loci (CRISPR nuclease and array) and the transposase proteins are located between the predicted transposon left and right ends (e.g. FIG. 4B and 4C). In this case, the effector was predicted to direct DNA integration to specific genomic locations based on a guide RNA.

In some systems, the effector was predicted to have homology with known CRISPR effector proteins, but to be inactive based on the absence of endonuclease domains and/or catalytic residues (FIG. 5A). The transposases were predicted to be associated with the effector when the CRISPR loci (inactive CRISPR nuclease and array) and the transposase proteins were located within the predicted transposon left and right ends (FIGS. 5A and 5B).

### Example 12—CAST Discovery

CRISPR-associated transposons (CAST) are systems that consist of a transposon that has evolved to interact with a CRISPR system to promote targeted integration of DNA cargo.

CASTs are genomic sequences encoding one or more protein sequences involved in DNA transposition within the signature left and right ends of the transposon. A Tn7 transposon, as defined here, consists of a catalytic transposase TnsB, but may also contain a catalytic transposase TnsA, a loader protein TnsC or TniB, and target recognition proteins TnsD, TnsE, TniQ, and/or other transposon-associated components. The transposon ends consist of predicted transposase binding sites, which contain direct and/or inverted repeats of 15 bp to 150 bp in length flanking the transposon machinery and other ‘cargo’ genes.

In addition, CASTs also encode a DNA and/or RNA targeting CRISPR nuclease or effector in the vicinity of a CRISPR array. In some systems, the effector was predicted to be an active nuclease based on the presence of endonuclease-associated catalytic domains and/or catalytic residues. In some systems, the effector was predicted to have sequence similarity with known CRISPR effector proteins, but to be inactive based on the absence of endonuclease domains and/or catalytic residues. The transposons were predicted to be associated with the effector when the CRISPR locus and the transposon-associated proteins were located within the predicted transposon left and right ends. In this case, the effector was predicted to direct DNA integration to specific genomic locations based on a guide RNA.

### Example 13—Class II Cas12K CAST

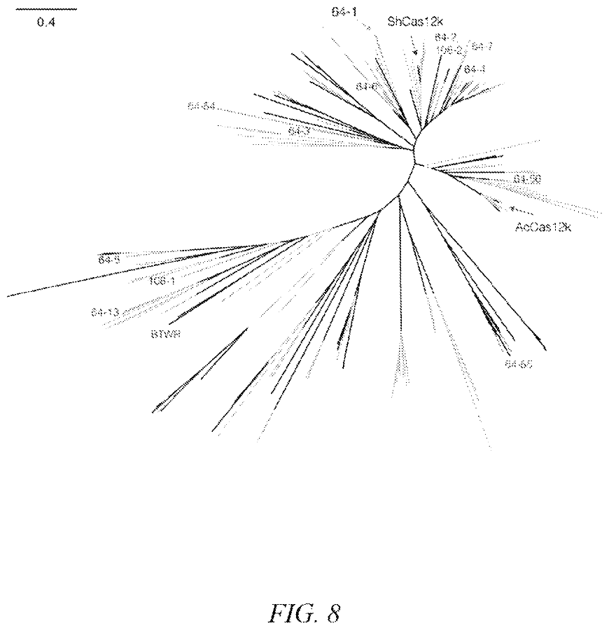

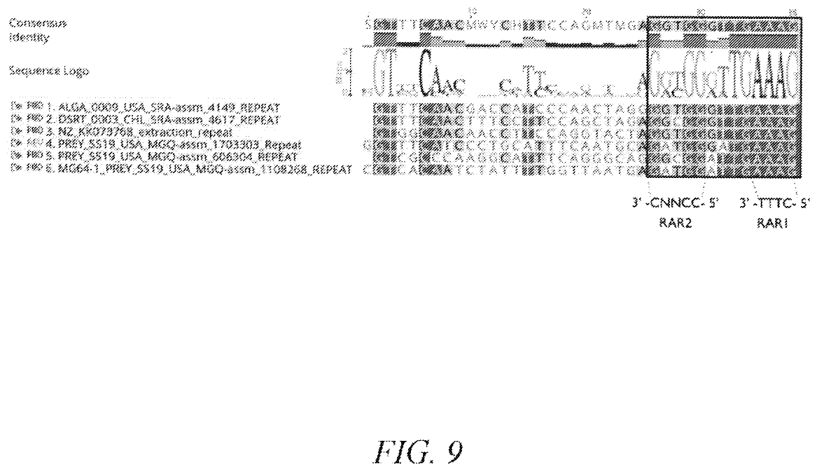

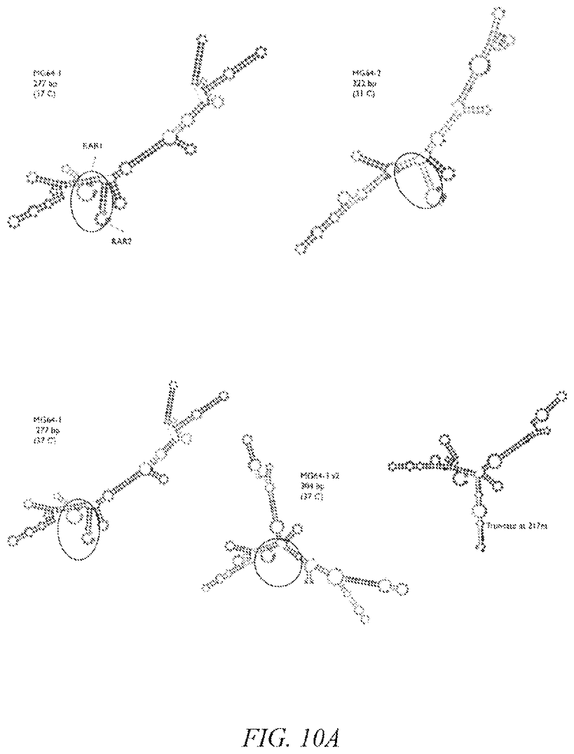

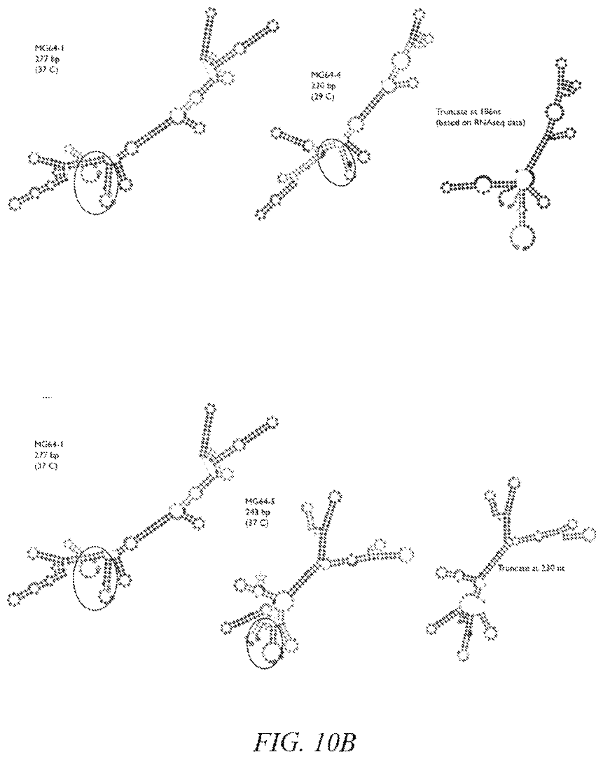

Cas12k CAST systems encode a nuclease-defective CRISPR Cas12k effector, a CRISPR array, a tracrRNA, and Tn7-like transposition proteins. Cas12k effectors are phylogenetically diverse and features that confirm their association with CASTs have been confirmed for several (FIG. 8). For example, the transposon left end was identified downstream from the MG64-3 CRISPR locus, as shown by terminal inverted repeats and self-matching spacer sequences (FIG. 11A). Cas12k CAST CRISPR repeats (crRNA) contain a conserved motif 5′-GNNGGNNTGAAAG-3′ (FIG. 9). Short repeat-antirepeats (RAR) within the crRNA motif aligned with different regions of the tracrRNA (FIG. 9 and FIGS. 10A and 10B), and RAR motifs appeared to define the start and end of the tracrRNA (For example, for MG64-1, the 5′ end of the tracrRNA contained RAR1 (TTTC) and the 3′ end contained RAR2 (CCNNC), (FIG. 10A).

### Example 14—Transposon End Prediction

Transposon ends were estimated from intergenic regions flanking the effector and the transposon machinery. For example, for Cas12k CAST, the intergenic region located directly upstream from TnsB and directly downstream from the CRISPR locus, were predicted as containing the Tn7 transposon left and right ends (LE and RE).

Direct and inverted repeats (DR/IR) of ˜12 bp were predicted on the contig, with up to 2 mismatches. In addition, the Dotplot algorithm was used to find short (˜10-20 bp) DR/IR flanking CAST transposons. Matching DR/IR located in intergenic regions flanking CAST effector and transposon genes are predicted to encode transposon binding sites. LE and RE extracted from intergenic regions, which encode putative transposon binding sites, were aligned to define the transposon ends boundaries. Putative transposon LE and RE ends are regions: a) located within 400 bp upstream and downstream from the first and last predicted transposon encoded genes; b) sharing multiple short inverted repeats; and c) sharing >65% nucleotide id.

### Example 15—Single Guide Design

Analysis of the intergenic regions surrounding the Cas effector and CRISPR array identified a potential anti-repeat sequence and a conserved “CYCC(n6)GGRG” stem loop structure neighboring the antirepeat corresponding to the duplexing sequence of the tracrRNA (FIG. 11B). TracrRNA and crRNA repeat were folded and trimmed, adding a tetraloop sequence of GAAA to maintain the stem loop region of the crRNA-tracrRNA complementary sequence.

### Example 16—In vitro Integration Activity Using Targeted Nuclease

In situ expression and protein sequence analyses indicated that some RNA guided effectors are active nucleases. They contain predicted endonuclease-associated domains (matching RuvC and HNH_endonuclease domains), and/or predicted HNH and RuvC catalytic residues. Candidate activity was tested with engineered single guide RNA sequences using the myTXTL system and in vitro transcribed RNA. Active proteins that successfully cleaved the library yielded a band around 170 bp in the gel.

### Example 17—Programmable DNA Integration

CAST activity was tested with five types of components (1) a Cas effector protein (SEQ ID NO: 1) expressed by myTXTL or PURExpress, (2) a target DNA fragment or plasmid containing the target sequence and PAM corresponding to the Cas enzyme (SEQ ID NO: 31), (3) a donor DNA fragment containing a marker or fragment of DNA flanked by the LE and RE of the transposase system in a DNA fragment or plasmid (SEQ ID NOs: 8-11) (4) any combination of transposase proteins expressed using myTXTL or PURExpress (SEQ ID NO: 2-4), and (5) an engineered in vitro transcribed single guide RNA sequence (SEQ ID NO: 5). Active systems that successfully transposed the donor fragment were assayed by PCR amplification of the donor-target junction.

After performing the transposition reaction, PCR amplification of the junction showed that proper donor-target formation occurred and that the transposition reaction was sg dependent. (FIG. 9). PCR amplification of reactions #3 and #4 indicated that both orientations of the donor relative to the target were made: one where the LE is closer to the PAM, and one where the RE is closer to the PAM. While both transposition orientations occurred, there appeared to be a preference for donor integration in the target where the LE is closer to the PAM, represented by strong band present for reactions #4 and #5.

Sanger sequencing of the preferred orientation product was performed. Of the integrations that occurred with the LE closer to the PAM, there was a clear degradation of the sequencing chromatogram signal from either the forward or reverse direction over the target/donor junction. This indicated that, of the products that were oriented with the LE closer to the PAM, integration occurred in a range of nucleotides, with the primary product of LE-closer-to-PAM products as a 61 bp integration from the PAM (FIG. 10A). Sequencing that originated from the donor over the donor-target junction defined the composition of the essential outer bounds of the LE and RE sequences (FIGS. 10A and 10B). Sequencing of the RE on LE-closer-to-PAM products showed a 3 bp duplication downstream of the donor RE (FIG. 10B). This is in part due to the Tn7 transposase integration event that cleaved and ligated the donor fragment at a staggered cut site. A 3 bp duplication is smaller than the expected 5 bp of duplication from other Tn7 transposases.

Sanger sequencing of the PCR amplified product over the 8N library of the target plasmid also indicated that the PAM preference of the MG64-1 effector as a nGTn/nGTt on the 5′ end of the spacer (FIG. 7C). NGS analysis of the PAM library target corroborated that the nGTn motif preference at the 5′ end.

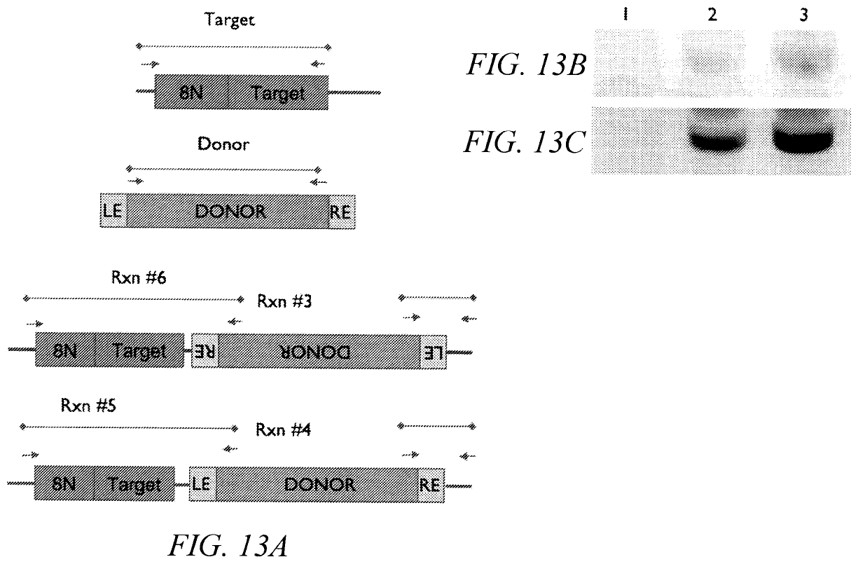

Further development of single guide testing confirmed activity of MG64-1 with a new sgRNA scaffold (FIGS. 13A-13C).

### Example 18—Integration Window Determination

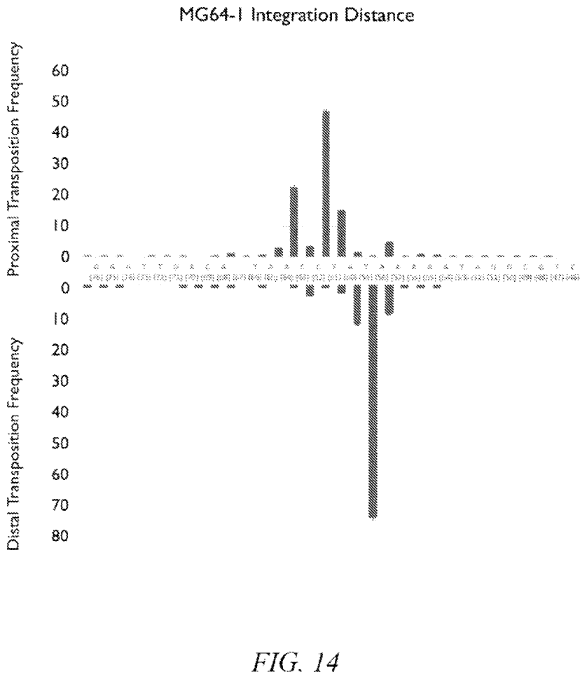

PCR junctions of the PAM that were amplified were indexed for NGS libraries and sequenced on a MiSeq with a V2 300 read kit. Reads were mapped and quantified using CRISPResso using an amplicon sequence of a putative transposition sequence with a 60 bp distance of integration from the PAM (guideseq=20 bp 3′ end of LE or RE, center of window=0, window size=20) Indel histogram was normalized to total indel reads detected, and frequencies were plotted relative to the 60 bp reference sequence (FIG. 14)

Both PCR reactions 5 (LE proximal to PAM, FIG. 14 top panel) and PCR 4 (RE distal to PAM, FIG. 14 bottom panel) were plotted on the sequence and distance from the PAM for MG64-1. Analysis of the integration window indicates that 95% of the integrations that occurred at the spacer PAM site were within a 10 bp window between 58 and 68 nucleotides away from the PAM. Differences in the integration distance between the distal and the proximal frequencies reflected the integration site duplication—a 3-5 base pair duplication as a result of staggered nuclease activity of the transposase upon integration.

### Example 19—Colony PCR Screen of Transposase Activity

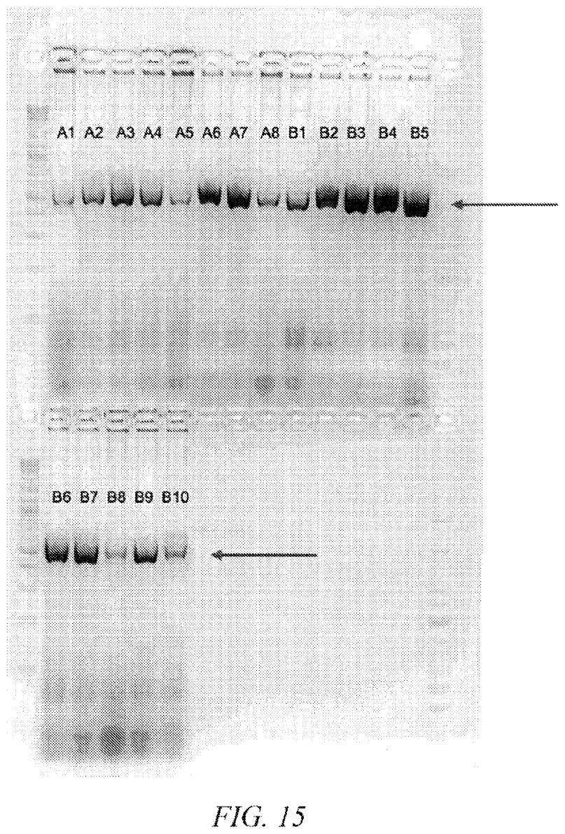

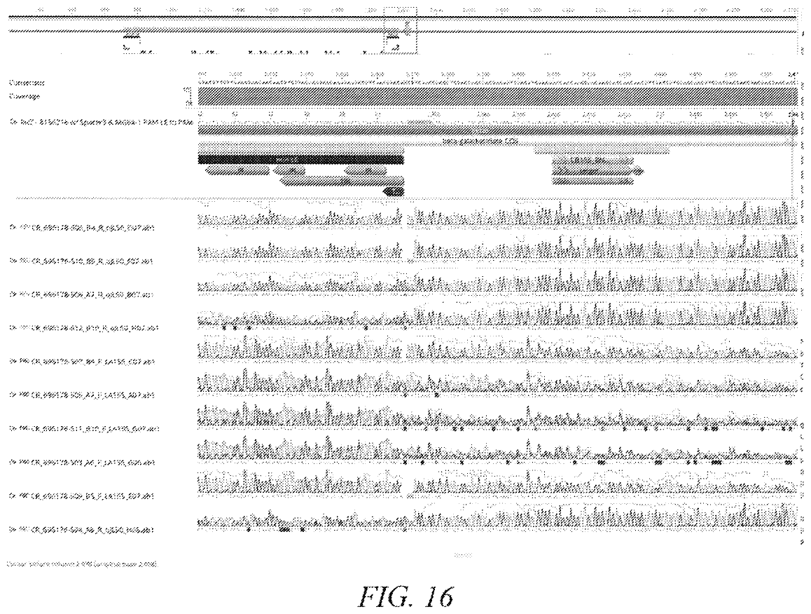

Transposition activity was assayed via a colony PCR screen. After transformation with the pDonor plasmids, E. coli were plated onto LB-agar containing ampicillin, chloramphenicol, and tetracycline. Select CFUs were added to a solution containing PCR reagents and primers that flank the selected insertion junction. PCR reactions of the integration products were visible on a gel (FIG. 15). Sequencing results of select colony PCR products confirmed that they represent transposition events, as they spanned the junction between the LE and the PAM at the engineered target site, which is in the lacZ gene (FIG. 16).

### Example 20—Single Guide Engineering

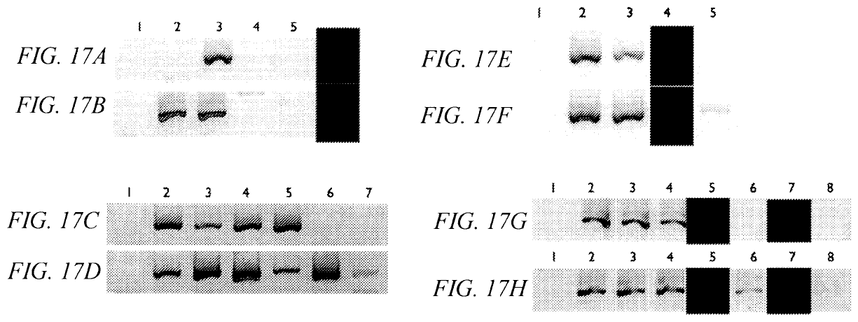

Predicted RNA folding of the active single RNA sequence was computed at 37° using the method of Andronescu 2007. All hairpin-loop secondary structures were single deleted from the construct and iteratively compiled into a smaller single guide. Engineered single guides (esg) 4, 6, 7, 8, 9 were active for donor transposition (FIGS. 17C and 17D), with engineered sgRNAs 8 and 9 being weaker single guides and transposing with PCRS (FIG. 17D). Engineered guide 5 was able to transpose, however engineered sgRNA 10 weakly transposed with PCR 5 (FIGS. 17E and 17F) Esg 17 is a combination of deletions in esg6 and esg7, and esg 18 is a combination of esg 4 and esg5. Both were able to strongly transpose across both PCR4 and 5 (FIGS. 17G and 17H), However, combinatorial addition of esg 6 and esg 18 making esg 19, resulted in a weaker transposition in PCRS, and addition of esg 7 to esg 19, making esg 20 results in a very weak junction of transposition for PCR 5 (FIGS. 17G and 17H). In a second approach, the tracrRNA of MG64-1 was aligned to known type Vk tracrRNA, and areas of unique insertions were mutated out of the single guide. sgRNA was minimized by truncation of insertion sequences of the MG64-1 sgRNA (FIG. 14). 2 subsequent deletions, esg 2 and esg 3 were also tested (FIGS. 17A and 17B) but neither esg2 nor esg3 resulted in appreciable transposition, thus the , and single guide was minimized by 57 bases.

### Example 21—LE-RE Minimization

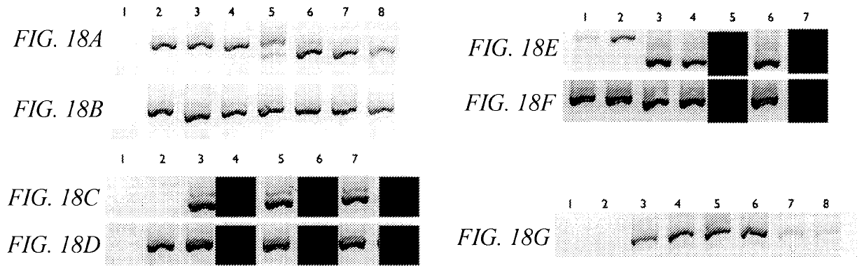

Sequencing of the target-transposition junction aided in identification of the terminal inverted repeats by identifying the outmost sequence from the donor plasmid that was incorporated into the target reaction. By performing repeat analysis of 14 bp with variability of 10%, short repeats contained within the terminal ends were identified and truncations of these minimal ends to preserve the repeats while deleting superfluous sequence were designed. Prediction and cloning was done in multiple iterations, with each interaction tested with in vitro transposition. Initial LE and RE deletions were singly designed and cloned to the 68 bp, 86 bp, and 105 bp for the LE, 178 bp, 196 bp and 242 bp for the RE. The RE of 64-1 also had a noticeable span of sequence without a repeat, so internal deletions of both 50 bp and 81 bp were designed and cloned. Transposition among all single deletions was robust for both PCR 4 and PCR 5 (FIGS. 18A and 18B) and internal deletion of 81 bp was subsequently pursued with combinatorial deletions for the RE. Trimmed ends of the former 178, 196 and 212 bp were cloned on the 81 bp internal deletion and transposition was tested. Transposition was active for all constructs designed. In combination with LE of 68 bp, we were determined that transposition proved active down to a LE region of 68 bp combined with a RE region of 96 bp (FIGS. 18E and 18F).

### Example 22—Overhang Influence of Transposition

In order to test whether superfluous sequence outside of the TnsB binding motifs were necessary for transposition, oligos designed for the TGTACA motifs of both LE and RE were designed and synthesized with 0, 1, 2, 3, 5 and 10 bp extra base pairs. These synthesized oligos were used to generate donor PCR fragments with overhangs and tested for their ability to transpose into the target site. Most noticeably, PCR6 was rarely detected from the in vitro reactions, (FIG. 18G lanes 1,2) however with a small 0-3 bp overhang, we were able to detect efficient integration at PCR 6, reflecting a RE proximal to PAM orientation that is not detected with a larger flanking sequence.

### Example 23—CAST NLS Design

Eukaryotic genome editing for therapeutic purposes is largely dependent on the import of editing enzymes into the nucleus. Small polypeptide stretches of larger proteins signal to cellular components for protein import across the nuclear membrane. Placement of these tags is not trivial, as these NLS tags need to provide import function while also maintaining function of the protein to which it is fused. In order to test functional orientations of the NLS to each of the components of the CAST complex, we designed and synthesized constructs fusing Nucleoplasmin NLS to the N-terminus and SV40 NLS to the C-terminus of each of the components of the MG CAST. Protein of these constructs were expressed in cell free in vitro transcription/translation reactions and tested for in vitro transposition activity with a complement set of untagged components. NLS-tagged constructs were assessed for maintenance of activity by PCR of the donor-target junction using PCR 4 (Assessing RE distal transpositions) and the cognate transposition event, PCR 5(LE to proximal transposition).

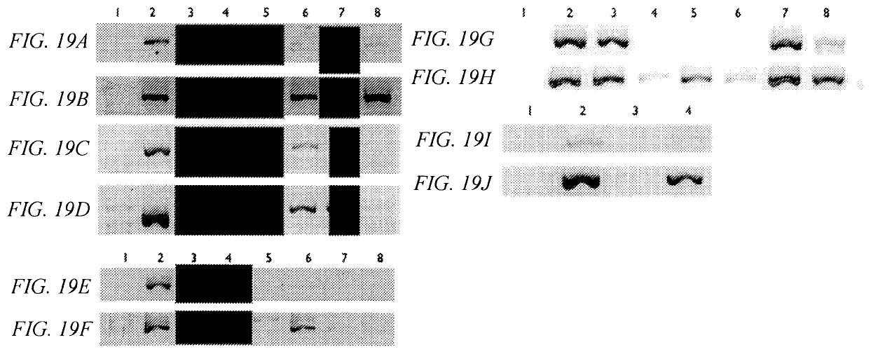

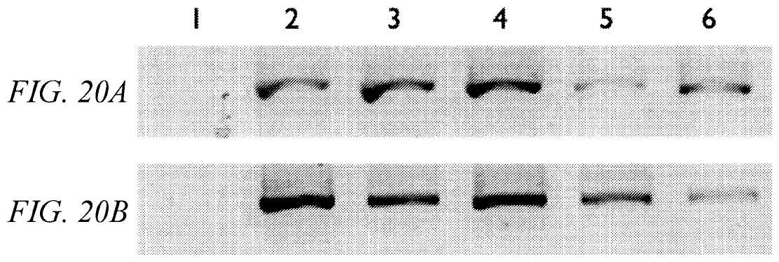

Most components resulted in a single NLS orientation that maintained activity. TnsB was the CAST component that was active with both N-terminal NLS and C terminal NLS by both PCR4 and PCR 5 (FIGS. 19A and 19B). TniQ was active with N-terminal NLS tags (FIGS. 19C and 19D). And Cas12k component was active with a C-terminal tagged NLS (FIGS. 19E and 19F, lanes 5,6). Further development of a Cas12k with both Nucleoplasmin and SV40 NLS tags were tested and found to be active (FIGS. 19I and 19J, Lane 4). TnsC was weakly active with an N-terminal NLS (FIGS. 19E and 19F, lane 7), but further exploration of the TnsC tagging identified new working NLS-HA-TnsC and NLS-FLAG-TnsC constructs (FIGS. 19G and 19H, lanes 3 and 7, respectively). The end result was a completely NLS-tagged suite of components that were active in vitro with both orientations of NLS-TnsB and TnsB-NLS (FIGS. 20A and 20B, lanes 5,6).

### Example 24—Cas12k and TniQ Protein Fusion Construct Design and Testing

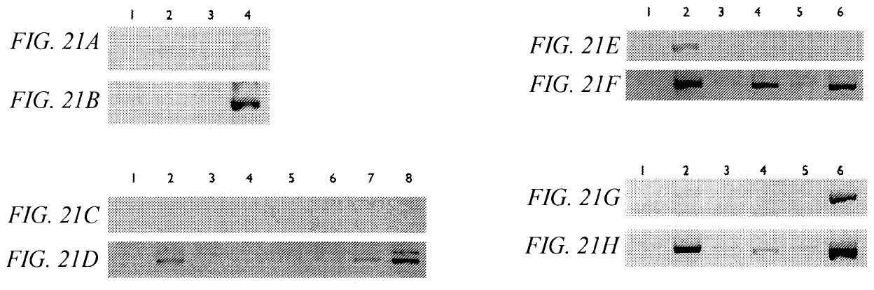

In an effort to simplify the expression of the protein components and minimize delivery of these components into cells, we designed, synthesized, and tested fusion constructs between the Cas12k effector and the TniQ protein. Both orientations of the TniQ fused to the Cas12k were designed and synthesized, a C-terminal fusion, Cas-TniQ, and an N terminal fusion, TniQ-Cas. While both constructs were weakly active for PCR4 (FIG. 21A), when expressed in vitro and assayed for transposition abilities, PCRS junction was robustly formed by the TniQ-Cas fusion protein (FIG. 21B). Transpositions lengths were assayed with variable linker domains including the original (20 amino acid linker), 48, 68 72 and 77 (FIGS. 21C-21F). NLS tags were then linked to the N terminus of TniQ and the C terminus of the Cas12k and found to still be active by PCRS (FIGS. 20E and 20F).

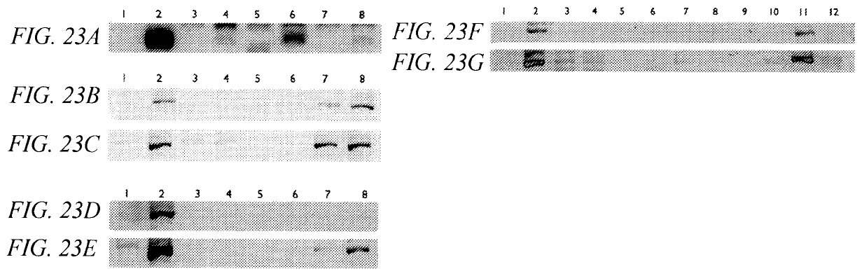

Two other linkers were employed to fuse the effector and TniQ genes. P2A, a self-stopping translation sequence was active in a Cas-NLS-P2A-NLS-TniQ construct (FIGS. 21G and 21H, lane 6), and an MCV Internal Ribosome Entry Sequence (IRES) mRNA-based linker allowed for independent translation of the two components in cells (FIGS. 23F and 23G).

### Example 25—Intracellular Expression Coupled in Vitro Transposition Testing

To test the functionality of the NLS constructs in a physiologically relevant environment, constructs cloned with active NLS-tagged CAST components were integrated into K562 cells using lentiviral transduction. Briefly, constructs cloned into lentiviral transfer plasmids were transfected into 293T cells with envelope and packaging plasmids, and virus containing supernatant was harvested from the media after 72 hr incubation. Media containing virus was then incubated with K562 cell lines with 8 μg/mL of polybrene for 72 hrs, and transfected cells were then selected for integration in bulk using Puromycin at 1 μg/mL for 4 days. Cell lines undergoing selection were harvested at the end of 4 days, and differentially lysed for nuclear and cytoplasmic fractions. Subsequent fractions were then tested for transposition capability with a complementary set of in vitro expressed components.

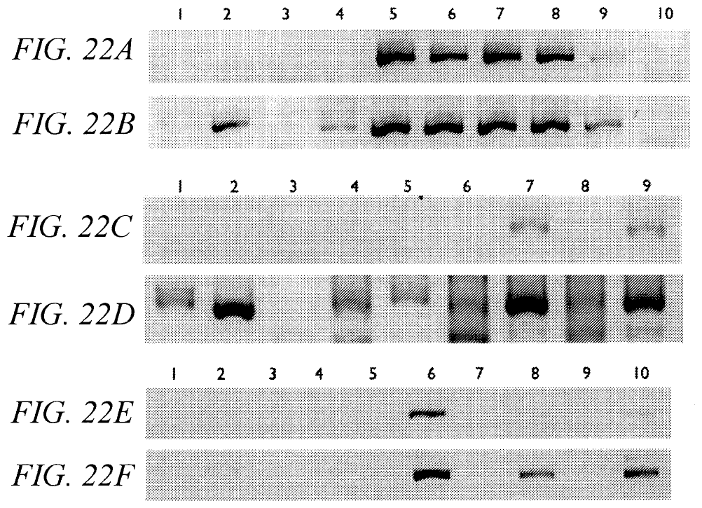

Both NLS-TnsB and TnsB-NLS were tested by cell fractionation and in vitro transposition, and transposition was detected across both cytoplasmic and nuclear fractions, and NLS-TniQ had detectable activity in the cytoplasm (FIGS. 22A and 22B). NLS-HA-TnsC and NLS-FLAG-TnsC were both active in both cytoplasmic and nuclear fractions when expressed (FIG. 22D), however PCR4 is formed in the nuclear fraction of both TnsC constructs. (FIG. 22C).

When both NLS-TnsB or TnsB-NLS were linked with NLS-FLAG-TnsC by using an IRES, NLS-TnsB-IRES-NLS-FLAG-TnsC was largely active in the nuclear fraction while TnsB-NLS-IRES-NLS-FLAG-TnsC was active in both cytoplasmic and nuclear fractions. This is indicative that NLS-TnsB has a higher capacity of trafficking to the nucleus (FIGS. 21E and 21F).

Cas12k fusions in the cell were similarly fractionated and tested for transposition. Cas-NLS Cas-NLS-P2A-NLS-TniQ were transduced into cells, fractionated, and tested in vitro for subcellular activity. Cas-NLS-P2A-NLS-TniQ was able to transpose in the cytoplasm with the addition of single guide to the reaction (FIG. 23A). By supplementing holo Cas protein (+sgRNA) or additional TniQ with sgRNA, we were able to complement the Cas-NLS-P2A-NLS-TniQ construct in the nuclear fraction. This indicates that both Cas-NLS and NLS-TniQ are making it into the nucleus (FIGS. 23B and 23C). NLS-TniQ-Cas-NLS fusion protein had similar results, but needed more supplementation with TniQ (FIGS. 23D and 23E), and Cas-NLS-IRES-NLS-TniQ needed supplementation from just the holo Cas-NLS (FIGS. 23F and 23G) As a whole this indicates that all the components of the CAST have been able to be delivered to the nuclear fraction of the cell.

### Example 26—Transposon End Verification Via Gel Shift

In order to verify the activity of TnsB on the predicted transposon end sequence, the LE of MG64-1 was amplified using FAM labeled oligos. MG64-1 TnsB protein was expressed using a cell free transcription/translation system and incubated with the LE FAM labeled product. After incubation for 30 minutes, binding was observed on a native 5% TBE gel (FIG. 24). Multiple bands of fluorescent product within the co-incubated lane (FIG. 24, lane 3) indicated a minimum of 2 TnsB binding sites.

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g. via cleavage of reverse-transcribed viral RNA or an amplified DNA sequence encoding a disease-causing mutation); as deactivated enzymes in combination with a probe to target and detect a specific nucleotide sequence (e.g. sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-56. (canceled)

57. An engineered nuclease system comprising:
an endonuclease comprising a RuvC domain, wherein said endonuclease is derived from an uncultivated microorganism, and wherein said endonuclease is a Class II, type V-K Cas effector having at least 80% sequence identity to any one of SEQ ID NO: 1, 12, 16, 20-30, 64, or 80-85; and
an engineered guide ribonucleic acid (RNA), wherein said engineered guide RNA is configured to form a complex with said endonuclease and said engineered guide RNA comprises a spacer sequence configured to hybridize to a target nucleic acid sequence.

58. The engineered nuclease system of claim 57, wherein said endonuclease comprises a sequence having at least 90% sequence identity to SEQ ID NO: 1.

59. The engineered nuclease system of claim 57, wherein said engineered guide RNA comprises a sequence comprising at least about 46-80 consecutive nucleotides having at least 80% sequence identity to SEQ ID NO: 5.

60. The engineered nuclease system of claim 57, wherein said engineered guide RNA comprises a sequence comprising at least about 46-80 consecutive nucleotides having at least 80% sequence identity to SEQ ID NO: 6.

61. The engineered nuclease system of claim 57, wherein said engineered guide RNA comprises a sequence having at least 80% sequence identity to non-degenerate nucleotides of SEQ ID NO: 106.

62. The engineered nuclease system of claim 57, wherein said engineered guide RNA comprises a sequence having at least 80% sequence identity to non-degenerate nucleotides of any one of SEQ ID NOs: 5, 45-63, 68-73, 96-101 or a variant thereof.

63. The engineered nuclease system of claim 57, wherein said endonuclease is configured to bind to a protospacer adjacent motif (PAM) sequence, wherein said PAM sequence comprises SEQ ID NO: 31.

64. A system for transposing a cargo nucleotide sequence to a target nucleic acid site comprising:
a first double-stranded nucleic acid comprising said cargo nucleotide sequence configured to interact with a Tn7 type transposase complex;
a Cas effector complex comprising a class II, type V Cas effector and an engineered guide polynucleotide configured to hybridize to said target nucleic acid site; and
said Tn7 type transposase complex, wherein said Tn7 type transposase complex is configured to bind said Cas effector complex, wherein said Tn7 type transposase complex comprises a TnsB subunit.

65. The system of claim 64, wherein said cargo nucleotide sequence is flanked by a left-hand transposase recognition sequence and a right-hand transposase recognition sequence.

66. The system of claim 64, wherein said class II, type V Cas effector comprises a polypeptide comprising a sequence having at least 80% sequence identity to any one of SEQ ID NO: 1, 12, 16, 20-30, 64, or 80-85, or a variant thereof.

67. The system of claim 66, wherein said class II, type V Cas effector comprises a polypeptide comprising a sequence having at least 90% sequence identity to SEQ ID NO: 1.

68. The system of claim 64, wherein said TnsB subunit comprises a polypeptide having a sequence having at least 80% sequence identity to any one of SEQ ID NOs: 2, 13, 17, or 65, or a variant thereof.

69. The system of claim 68, wherein said TnsB subunit comprises a polypeptide having a sequence having at least 90% sequence identity to SEQ ID NO: 2.

70. The system of claim 64, wherein said Tn7 type transposase complex comprises a polypeptide comprising a sequence having at least 80% sequence identity to any one of SEQ ID NOs: 3-4, 14-15, 18-19, or 66-67, ora variant thereof.

71. The system of claim 70, wherein said Tn7 type transposase complex comprises a polypeptide comprising a sequence having at least 90% sequence identity to any one of SEQ ID NOs: 3 or 4.

72. The system of claim 64, wherein said engineered guide polynucleotide comprises a sequence comprising at least about 46-80 consecutive nucleotides having at least 80% sequence identity to any one of SEQ ID NOs: 5-6, 32-33, 94-95, or 104-105, or a variant thereof.

73. The system of claim 72, wherein said engineered guide polynucleotide comprises a sequence comprising at least about 46-80 consecutive nucleotides having at least 90% sequence identity to any one of SEQ ID NOs: 5 or 6.

74. The system of claim 64, wherein said engineered guide polynucleotide comprises a sequence having at least 80% sequence identity to non-degenerate nucleotides of any one of SEQ ID NOs: 106, 107, 108, 5, 45-63, 68-75, or 96-103, or a variant thereof.

75. The system of claim 65, wherein said left-hand transposase recognition sequence comprises a sequence having at least 80% sequence identity to any one of SEQ ID NOs: 9, 11, 36-38, 76, or 78, or a variant thereof.

76. The system of claim 65, wherein said right-hand transposase recognition sequence comprises a sequence having at least 80% sequence identity to any one of SEQ ID NOs: 8, 10, 39-44, 77, 79, or 93, or a variant thereof.


SEQUENCE LISTING (108 sequences)

SEQ ID NO: 1 - protein, 612 aa, unidentified
  REGION 1..612: note = Description of Unknown: MG64 active effector sequence
  REGION 1..612: note = MG64-1 active effector
MSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 2 - protein, 563 aa, unidentified
  REGION 1..563: note = Description of Unknown: MG64 active transposition protein sequence
  REGION 1..563: note = MG64-1-B active transposition protein
MKNANSPPSSSVDDHQKEQHLVIPSELSDEAQLKLEVIQTLLEPCDRRTYGQRLREAAEKLGKSKRTVQRLVKKWEEEGLEAIAPTNRSDKGDFRIEEQLQEFIIKTYQNGNKGSLRVTRKQVYLKTKAKAEELSINPPSHMTVYRILQPLIEKQEKKKSIRSPGWRGSQLSVKTRAGQDLSVEYSNHVWQCDHTRADLLLVDQYGELLGRPWLTTVIDTYSRCIIGINLGFDAPSSQVVALALRHAILPKYYTPDYLLGEEWGTYGKPEHFYTDGGKDFRSNHLQQISVQLGFVCHLRDRPSEGGIVERPFKTLNLEFFSTLPGYTGSNVQERPEDAEKEACLTLRQLEQKLVRYIVDNYNQRMDARMGDQTRFQRWESGLIATPDVISERELDICLMKQTRRKVQRGGYLQFENLMYRGENLAGYAGESVILRFDPRDITTVLVYQKESNHEVFLTRAYAMDLETEQMSLDEAKASSKRVRDAGKTVGNRSILSEVRDRQIFPKAKKSKKERYQEEQKAITSKPLEITEWESEETDFSPPSSETPQVEVFDYETLQEDYGF

SEQ ID NO: 3 - protein, 278 aa, unidentified
  REGION 1..278: note = Description of Unknown: MG64 active transposition protein sequence
  REGION 1..278: note = MG64-1-C active transposition protein
MTIQEAQAVAKQLGDIQLTSEKLQAEIQRLNRKTVVTLSHVEALHNWLEGKRQAKQSCRVVGESRTGKTIACNAYRLRHKPIQTPGKPPIVPVVYIQVTQECGAKDLFGAIIEHLKYQMTKGTVAEIRQRTFKVLQRCGVEMLIIDEADRLKPKTFAEVRDIFDKLNIAVVLVGTDRLDAVIKRDEQVYNRFRACHRFGKLAGDEFSQTVNIWERQVLKLPVASNLSSKRMLKILGQATGGYLGLLDMILRESAIRALKKGLQKIDLDTLKEVTEEYR

SEQ ID NO: 4 - protein, 171 aa, unidentified
  REGION 1..171: note = Description of Unknown: MG64 active transposition protein sequence
  REGION 1..171: note = MG64-1-Q active transposition protein
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYL

SEQ ID NO: 5 - DNA, 319 bp, synthetic construct
  misc_feature 1..319: note = Description of Artificial Sequence: Synthetic polynucleotide
  misc_feature 1..319: note = MG64-1 active effector tested sgRNA sequence
  misc_difference 296..319: note = any nucleotide
  source 1..319: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaagnnnnnnnnnnnnnnnnnnnnnnnn

SEQ ID NO: 6 - DNA, 277 bp, synthetic construct
  misc_feature 1..277: note = Description of Artificial Sequence: Synthetic polynucleotide
  misc_feature 1..277: note = MG64-1 active effector tracrRNA sequence
  source 1..277: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccact

SEQ ID NO: 7 - DNA, 38 bp, synthetic construct
  misc_feature 1..38: note = Description of Artificial Sequence: Synthetic oligonucleotide
  misc_feature 1..38: note = MG64-1 active effector's target CRISPR repeat
  source 1..38: mol_type = other DNA
cgtcacaatctattttggttaatgagatggattgaaag

SEQ ID NO: 8 - DNA, 449 bp, synthetic construct
  misc_feature 1..449: note = Description of Artificial Sequence: Synthetic polynucleotide
  misc_feature 1..449: note = MG64-1 active transposon end RE
  source 1..449: mol_type = other DNA
aatgaacaaaaatgtctgattattacataattgtttatttaatataattgtatcgtaatacttgaagtttggagacaagtaatttgttaatactgctccagtccctaaaaaagtgccattcgggtaaatgacacttaatctgttaatttactggaaaatgacagttaatttgttaatatagtaagcaataacttttgtcaaagattaatgctataattcagctaaagcagtgattatataaagctttcactctcaaatagttcggcgacacgattttgttaagacgacaaataattagttactgtacatttacccataactttgccgttttggtaaggttatcgttcaaaacacaagtggcaagattatggttcaaaacctaagtcccgtttagtttgcttgaacacttcacgaacttgagggtaacgaagaaagctagggtgagtcaa

SEQ ID NO: 9 - DNA, 319 bp, synthetic construct
  misc_feature 1..319: note = Description of Artificial Sequence: Synthetic polynucleotide
  misc_feature 1..319: note = MG64-1 active transposon end LE
  source 1..319: mol_type = other DNA
ctcttaaggaaatctatgtaagtttgttgggttagttgcgttttcagtaaatactgtgttatagtaagaacttgtgcggacgtatagctcagttggttagagtacatcgttgacatcgatggggtcactggttcgagtccagttacgtccatatttttttgaagtgtgtataatattaactatgtgactttatgtacattaacagattatttgtcatcggtaacaaattgttgtcatcttaacaaaatatttgtcatcaataacatattatgtgtcgtgtgcttattactgaaactaatcctagacgatggtaaaaaat

SEQ ID NO: 10 - DNA, 308 bp, synthetic construct
  misc_feature 1..308: note = Description of Artificial Sequence: Synthetic polynucleotide
  misc_feature 1..308: note = MG64-1 active transposon outer bounds of RE
  source 1..308: mol_type = other DNA
tgtacagtaactaattatttgtcgtcttaacaaaatcgtgtcgccgaactatttgagagtgaaagctttatataatcactgctttagctgaattatagcattaatctttgacaaaagttattgcttactatattaacaaattaactgtcattttccagtaaattaacagattaagtgtcatttacccgaatggcacttttttagggactggagcagtattaacaaattacttgtctccaaacttcaagtattacgatacaattatattaaataaacaattatgtaataatcagacatttttgttcatt

SEQ ID NO: 11 - DNA, 127 bp, synthetic construct
  misc_feature 1..127: note = Description of Artificial Sequence: Synthetic polynucleotide
  misc_feature 1..127: note = MG64-1 active transposon minimal LE
  source 1..127: mol_type = other DNA
tgtacattaacagattatttgtcatcggtaacaaattgttgtcatcttaacaaaatatttgtcatcaataacatattatgtgtcgtgtgcttattactgaaactaatcctagacgatggtaaaaaat

SEQ ID NO: 12 - protein, 643 aa, unidentified
  REGION 1..643: note = Description of Unknown: MG64 effector sequence
  REGION 1..643: note = MG64-3 effector
MSKITIQCRLVASEATRQYLWHLMADIYTPFVNEMLRQIREDDNFEQWRQAGKIPAGVFEDYRKALKTESRFQGMPGRWYYAGREEVKRIYKSWLALRRRLRNQLSGQNRWLEVLQSDETLMAVSGLDLPALRAASTKLLDLLSSQIAKPAKGSKKTNRGKGKKQAKQTQGKSLYQSLWDLYKETEDILQKCAIAYLLKNKSQVPDKPEDPEKFRHRRRKAEIRTERLNEQLTKTRLPKGRDLTNEQWLEALAIATEQIPKDETEAAIWQSRLLTDAASLPFPVAYETNEDLKWFLNGKGRLCVSFNGLSEHTFEIYCDKRQLHWFKRFLEDQQIKKEHQGKRSSGLFTLRSGRISWTSPSDIDKSPCWTANRLTLHCSVDTRLWTQEGTEEVRQEKATNIAKIIAGTKAKGNLNQKQQDFITKRETTLKLLHNPFPRPSKPLYQGNPSIIAAVSFGLEKPATLAIVDITTGKAITYRSIRQLLDQNYKLFTKHRLQQQQRAHQRHQNQKESAENRISEGGLGEHVDSLIAKAILETAAEYGASSIVLPELGNIREIIQAEVIAKAERKIPGLKEKQDEYAAKFRASVHRWSYGRLAQKITTKAALQGLETESTRQPLQGSPQEKARNLAIAAYESRKVDQRA

SEQ ID NO: 13 - protein, 542 aa, unidentified
  REGION 1..542: note = Description of Unknown: MG64 transposition protein sequence
  REGION 1..542: note = MG64-3-B transposition protein
MELVNPDDLNSVEARLKLEIVEKLSEPCDRRTYGERLRSAAQKLECSVRTVQRLMKKWEQEGIHALIDSGRKDKGNPRISEDWQSFIKDAYDNGKCTPAQVFTKVRQRARQEGLDSHPSHMTVYRILNPLIEAKEQKNNIRNVGWKGARLALKTRDGEVLEIDYSNQVWQCDHTRADILLVDKYGYQMGRPWLTTVVDTYSRAIVGINLGYDAPSSRVVALALRHAILPKQYGAEYKLYAEWPTCGVPDHMFTDGGKDFRSNHLQQIGVQLGFICHLRDRPSEGGIVERPFGTINTQFFSTLPGYTGSNVQDRPPEAEAEACLTLHELEKLLVAYIVNTYNQRLDARMGDQTRIQRWEAGLLKQPPTISERELDICLMKQTRRTIYRGGYLQFENLTYWGETLAEQAGENIVLRYDPRDITRLLVYRYESDREVYLGVAQAQDLEGEVLALDDAKAHSRRIREDGKAVSNDAMLDEMRDREAFVDEKKKSRKERQKEEQEDLRQTPLPVIEADSFDEDESGEPQDNLEIPEFEIWEFDNNDI

SEQ ID NO: 14 - protein, 272 aa, unidentified
  REGION 1..272: note = Description of Unknown: MG64 transposition protein sequence
  REGION 1..272: note = MG64-3-C transposition protein
MVTTTLSEQEIQAHIERLRKDKTVALESVQQAHTWLDRKRNARQCGRIIGESRTGKTKACESYLKKNGLPDLSGKIPNIPISYFVPKQDCTSRELFRAILEHYGDELPRGTVGDARSKTFKVLRECQTEMLIIDEADRLKPKTFADVRDIFDNLEISVVLVGTKKRLDKVVKADEQVFNRFRSSYKIGTIPSSQLATIVGVWERDILKLPLPSNLTSESMLKEIRRATGKSRKGYYIGLIDMVLRAAAVMALEKGQMKVDKATLKVAVEDYL

SEQ ID NO: 15 - protein, 165 aa, unidentified
  REGION 1..165: note = Description of Unknown: MG64 transposition protein sequence
  REGION 1..165: note = MG64-3-Q transposition protein
MTMPIVPTWVFPVDPYSGESLSHFLGRFCRENHATLNQLGEKTGLGAVLGRWEKFRFIPPPNDAQLAALAKLVRLEVDQIKQMLPQEAMQNRVIRLCAACYAEEPYHRIEWQYKLANRCDRHHLLLLLECPNCKAKLPMPSKWANGTCKRCLTPFDQMVALQKGV

SEQ ID NO: 16 - protein, 692 aa, unidentified
  REGION 1..692: note = Description of Unknown: MG64 effector sequence
  REGION 1..692: note = MG64-5 effector
MKTIRCCLCANPETRRYFWKIMVTYTLLVNELLAAMPQRPEFAQWKQRGTIAREAVRIVLTPLKTDPTYADLPKRFFSSAELLVCYVYKSWLALQKRRYWKLVGKQRWLQVIEDDLQSLLTDNFSLESVQSKAHQILEQAHKELEKQPQRFKKKGKKSRPLFGYLLDLYGTTADKLERRAIGHLLHHDLKVSDTEDFPETIQFSIDQQQVEIARLKEQLQSRLPDGRDPTQARFLEKLRIATALPELELEGFDEEHFSEWRTQKQIPLLNPLPYPVLFGSSSDLHWKLEPQKATTEANISPEVPTARSERVKERIQVRFKGDELQDSWFKLQCDRRQLPIFRQFVTDYLCQKQAPDHEKFGEGLFTLRSACLVWKEDPQGARKRKKRRKQGACQDEPWETHRLYLHCTIDTRFLTQEGTEQVRATKLDLAQKALEGIENKTALETVTQEPSAEQQKHLKRKQTTVHRLETQKPPVRPTIQPYEGKSNIVVGVSLSRHEPVTLIVFDTAQNKVLECMGTQALLKIHGIQSPRKNRSIGKLQQEQSQLLRRWRRKRKQNPHRRADGQRQDNYRSGNSESKLGDYLDRLIAARLVALATKRQASVIVLPELGDIRESVECSLQAKAQRKYPQHKKLQAKYAKHFRHEFHRWSYGRLQQYIAERATQQNLALLKGRQPKQGTEQEKVLEIISSACL

SEQ ID NO: 17 - protein, 636 aa, unidentified
  REGION 1..636: note = Description of Unknown: MG64 transposition protein sequence
  REGION 1..636: note = MG64-5-B transposition protein
MTNSRLGGTMKNLSDHPDSEKLTYEQEHCLVDELSPELQRKVELIQAIVDAPDRKTERQRIAIAAQELGRCTKTIRSYRDALREDGIVALTRTERSDKGQRRNISQPWIDLVLALYKRGQRSFCRSRNQVWLLIQGMTSKLLSDDWKTPEKRAELMEWYAQKLGAAAENAKSKLNKILGSIRKELEVGICMPPRSHMSVYGIIDDYLEQQHRKARHPGQGPEQVIQTTGELLVIEVTNGIFQADHSGIDILLKDKDGNEIGYPFLTVIIECASGCVTGFYLGFRQPGSHEVALALRHAILPKQYGPEYKLEKQWQCVGIPRYLVTDRAKEFKSKHLQQIAAELGFELRYRAYPSQGGLVESVFDKINKEVLSNLPGYKGSNVQKRPKNAEKYACLTIEDLEQELVRYFCDHYNQHFYPRMKDRTRAMQWEERLVEPPVIPDERELDLCLLKRKQTAKVQKYGTIQFQNEIYQGNCLLGRETEKISFRYNPSNIIHVLAYTVEETDKPSKFLGVLKARDRKEEKLSLHSLKLEQKLIRARGKKLDQSSIYNDALKRNERAERELHGLRKQQRRKEHERTGRSEGLGNVIDFKRQENEAIGTKNSQIGPIQKLVKRLKPKRKAKVAAKNWQQKLSENW

SEQ ID NO: 18 - protein, 291 aa, unidentified
  REGION 1..291: note = Description of Unknown: MG64 transposition protein sequence
  REGION 1..291: note = MG64-5-C transposition protein
MAQPQLISQQLQTQPSPFPLPDKEAEIDRLRAGAPFLTTDRDTALEQWLDTQRKSGNPGFICSAKGSGLSDSCQDYRMNRVRSKGMIQQLPVPVVYVRVPPICSVSHFHTTLLTALNHPITTGRLKDKRPRVRGRLKSIQTRQLIIDDADFLSFEALSEIAQIYDDLKIPSILCGTYYLEKRLQQRYWDRIGNSFLDFYEYPPMSQDEVVEVIDTWETEFLQWPEESDLLIEDVLKAVYVKTGGLRDALNEVLRKVAIQALKQDSYKITTEIIVSVLNGRVQPRIKPAQEE

SEQ ID NO: 19 - protein, 169 aa, unidentified
  REGION 1..169: note = Description of Unknown: MG64 transposition protein sequence
  REGION 1..169: note = MG64-5-Q transposition protein
MTDQQTVWMEHVEPYEGESISHYFGRFRRVEGNSFSAPTTLSAAVGIGPALSRWEKFRFNPFPSPQELEAMGKLIGLTVEQLRTMLPAKGERLVMRSTRLCGACYREAPYHRIHWQYESTEGCDKHRLRLISRCPVCDEKFALPVEWIEGACKQCGMKFTSMHKKQKPY

SEQ ID NO: 20 - protein, 640 aa, unidentified
  REGION 1..640: note = Description of Unknown: MG64 effector sequence
  REGION 1..640: note = MG64-33 effector
MKESLYFVIRCLLSADAETRRTMWLLMQRYTLLVNKLLEVIPGSSEFPVWREQGYIPDNALAEFIDTIKPDLDYSGLPGRFYTSAKILVKNIYKSWFALQRKYSRKITGKIRWIKIINSEIDLIKNTEFELDQITNAANAALKLAKKKKEESEKSNSESSTSLLGILIEMQFKTKSPLKKRGINHLLLNNLNIEYKDFTLDSLEARVEVAFLEIEALEKRLRSRLPKGRDPDGYRYVLALSKAASLPEEALTPEKFDEIHADIPIYNELPYPLIYEGASNIVWTLIKPEGNRSNFGRLQIHFNGISELKFLIQCGRRQLPVFKGFYHDAIENKGRISRGEIPYNEGLNRFRSAQILWKPDPSLDFRKKKKNIPSTPWEVNRLYLHCSVDKATLSAEGTECLRQMKIKKIEEKKEKPLSPRKQTELERLQSAAPPPRPSIQPYVGDPDMVVCICFSPDEPVIVVPVDLAKEAALYALNTKALLNRATKAIWRMGKLETLSDNGKALCHDNGGKLNIRNPRTYSVQKPYGLVTRLNTLSEQQVKRRTREQSKGKYRGSQSLSNLSLSVCRLIAARLVDLSLQLNAGRVIIPDFEGIRDWVQAFIAAKAVKAFPDSKQQQKKFRQEFRAKYHRWSYRKLAQEI

SEQ ID NO: 21 - protein, 612 aa, unidentified; identical to SEQ ID NO: 1
  REGION 1..612: note = Description of Unknown: MG64 effector sequence
  REGION 1..612: note = MG64-34 effector

SEQ ID NO: 22 - protein, 525 aa, unidentified
  REGION 1..525: note = Description of Unknown: MG64 effector sequence
  REGION 1..525: note = MG64-35 effector
REVLSQLSTQSTIEGPPDTQTKRPKAKSRKSKKKQSTAQNKDLIGKLYKAYEATNDLTQRCILAYLIKDAGTISEEEETPEAFTHRIHRKQKDIARLEDRLQARLPKGRDLTGDIFTDTLFIAQHQEPEDVNQMRDWQAKLLMRPADLPDPIRYDSSTDMMWKPDDQGRITVNFNGLEKFLKNSDLEVKSWLKEHQAYPFRIQCDQRQLPYFQRFLADWQAYTADAENYPAGLLTLSSAMLAWRKGKKNRKGEPWNIHQLVLYCSFDTRLLTAEGTAAVQQQKIEKAQKQAESAQNKKLNDNQRQARNRSATTLRKLDNLPTRPSQKAYQAKPELLLGLSIGLSEPITVAVVDASTQQVLTYRTSHTLLGEQHRLLRRQRQKQQQNRLKRQQNQKKGIRHQPSESELGQYVDRLLAKAITQLAQSHQVSSIVLPNLLNRRDLLDSEIQARAEQQCPGSISAQEKYAKAFRQSLHSWDYRRLIEAIRGSAGKHDIPLEEAFLTASSDPKEQAKEIAIAAYQARTED

SEQ ID NO: 23 - protein, 724 aa, unidentified
  REGION 1..724: note = Description of Unknown: MG64 effector sequence
  REGION 1..724: note = MG64-36 effector
METREMSQPNLPACMKTIMCCLCASPETRRYFWETMVSYTLLVNELLEAVPNRPEFPQWQRRGTIDREAVRIVLKPLKAKPNYAQLPKRFFTSAELIVCYVYKSWLALQKRRQWQLEGKHRWLAAIASDLKSILSSDLSFETVQAKARQILEQAEQDLEPPPPEVTKKGKKSKRRKKSKSLLKYLLDRHDETTQELERRAICHLLRHDLKVIEEEDTPETIQHVIDRKRIEIERLTEQLQSRLPKGRDPNHERFMERLEMAIALPDGSPKHWDPEEFDEWRIQKQIPELNTLPYPILFGSASDLYWDILNDTTSAATVSAKKKSRKSKRPNERLQVRFKGLDEHKCKIQCDRRQLKTFRQFATDYISNQQLPKDEKFGEGLFALRSACLIWKVDPDASASRRNRQKAVLRKDSHLKASLEKGEVCLIDYPWETHRLYLHCTFDIRLLTQQGTEQVRLKKLDAAQKSVEKTQERQAADPSITMTANQASRFKAKQTSISRLEKNRPAERPECQIYQPNPNIVVGISLSRHEPVTVVVFNKEKNQASEYWSTESLLKMRGITSPRNNQSIVQLQHEQQQLLRRWRRQRHYNIYQRPEGQKQGDYHQHDAESRLGDYLDRLIAARVTELAVRRQAAAIALPELQNIRESVESDIQARAEKKHPHHANLQAQYAKQYRREFHRWSFGRFEQYITEAAKQRGIAVYKGRQPKHGNEQEKALAVVTNVIA

SEQ ID NO: 24 - protein, 618 aa, unidentified
  REGION 1..618: note = Description of Unknown: MG64 effector sequence
  REGION 1..618: note = MG64-37 effector
MSQKTVRARLIVPEETRKAFWELTAGDNTPLVNEALRLLPTHSDFSKWRQKGNLPDKIAEDLVKTLKEDLRFVGQPFWSYISAHKQVTYTFRSWLALQHRKQWKLAGKRLWLEILQPDEILAESVGYTPDALIKAAKKNLADIEAQDNPFDALFSAYRKTKSLKRKSAIAYLLKRSAKLLPEEEDIAKLAQRYRKTEIFIQRLEAQLKASLPKGRDMSGDRQLEALQQIIQAPPMDDVSYNAWKNALTTEPAAFPFPISIETAAWLIWSQDDKGRLLLQLSGWGQHTFKVYFDKAHQHWFWRFLQDQETNQNGGDQHSAALFTLRAAKIMWFPSKKHKDAPEPWHRYHLNLLCTIDTRAWTQEGTEIIAQEKAVKTAKQLASMRKKESLTQNQQGYIRRLESTLNRLQVPYPRPSRPIYQGKPEILVGVSMGLEKVATVAVVNALTGRVLTYRSEKQLLGENYPLLRQARAEIAKKSHQGHRQRLRGVKSISKESDKGKQVDRLFAKAIVELVVEHQAGSIVLPDLAYKREIIEAEFQQRAIEKVPDFVDGQKEYAKAYLSQVHRWPYARLQGCTTSKAEQSGISCEITKQQYSGTPQDKAKGLGFLAYSQRSTALAE

SEQ ID NO: 25 - protein, 661 aa, unidentified
  REGION 1..661: note = Description of Unknown: MG64 effector sequence
  REGION 1..661: note = MG64-38 effector
MSIITIHCHLHTTEAIRRLLWQVMAASNTPLISTLLRHVAEHPDFDTWQTNGSVPVKTVRNIAEPLKAHYPSQPGRFYASAYQMVSYTYESWLATQKMIKLRLDGTRRWLSIFKSDAELLELTGLSLESLRQSAREVLSQLSTQSTAGRPSDTQTKPPKAKSRKSKKKQATAQDKDLIGKLFKAYEATDDLTQRCILAYLIKNGGTITDEAETPEAFAHRLHRKQKDIAQLENRLQARLPKGRDLTGDTFIDTLLIAQQQEPEDVAQMRDWQAKLLMRPADLPYPIRYDSSTDMMWKPDDQERITVNFNGLEKFLKNSDPAVKAWLKEHKEYPFRIQCDQRQLPYFQRFLTDWQAYTADKANYPAGLLTLSSAMLAWRKSKKKRKGEPWNTYQLALYCSFDTRLLTAEGTVEVQQEKLRKAQKQANSTKDKKLDENQLQAQTRSATSLRKLENLPARPSRKPYEGKSELLLGISIGFSEPVTVAIVDASTQQAITYRTSRTLLGDQHRLLRRQRQQKQQNRLKRQQNQKKGIRHQPSESELGQYVDRLLAKAIIQLAQTHQVSSIVLPNLTNDRDILNSEIQARAEQKCPGAIAAQAKYAKEVRISIHSWDYRRLSDAIRSSASKQGIPLEEAFLTVRTNPKEQARELAIAAYQARTENRN

SEQ ID NO: 26 - protein, 625 aa, unidentified
  REGION 1..625: note = Description of Unknown: MG64 effector sequence
  REGION 1..625: note = MG64-39 effector
ASSTHKPMSQKTIRCRLVASEETRRAIWQLMAERNTPLVNEVLRQLPEHPDFPKWQQRGKLPDLPVKRLIDSLKPDPRFCDQPVWYYISAQKQVAYTFRSWLSLQKRKQWRLEGKRRWLDILQPDAELAEQAKCSVEALRLAASNMLKKVDDPDPFKLLFKEYGTSKSTKRQCALAYLLKRDAKLEPEAEDLEKLDQRRSKAEIQIKQLETQLKASLPKGRDLTGQIQAQALTQSVQSPPLDDEAYSTWHASLAREPAIFPFPIIYETIESLVWSKNSKGRYSVCFQGQGTGIHTFKIYCDKPHQHWFERFWIDQETKRSGNDRHSAGLFTLRSARLSWIPSKKHQDEPEPWNRYYLSLSCTVDTALWTQEGTQTVIQEKAVATASKLQSMQEKESLNKNQQGYVRRLESTLTRLQTPYPRPSRALYQGRSDILVGVSMGLDKPATVAVVNALTGEVLTYRSTKQLLGEQYPLLQRARSERAKVAHQGHRQRRKGGKRVNQESNLGKHVDRLLAKAIVEVAQQYQAGSIVLPDLAHIREIVESEVKQKAAKKVPDFLDGQKQYAKAYRTQVHQWSYHRLQDAITSKAGQSSIATEVAKQDYSGSPQEKAKSLCLAGYEQRLALSS

SEQ ID NO: 27 - protein, 595 aa, unidentified
  REGION 1..595: note = Description of Unknown: MG64 effector sequence
  REGION 1..595: note = MG64-40 effector
MAERNTPLVNEVLRQLPEHPDFAKWQQKGNLPDVAVKRIIDALKSDPHFSDQPFWYYTSAQKQVTYTFKSWLSIQRRKQWRLQGKRFWLEILLPDAKLAELAECSVEKLRTEAAKILTKVGDVDPFKHLLEQYRHEKKLLRKYAIAFLLKRNTGIDREEDLEQLKQRSRRVELQIRRLEIQLQASLPKGRDLTGERQAAALAQSVLASPDDDESYELWRNTVTREPAQFPFPVICETSEWLKWQRDQNGRISVGFSALSEHVFKIYCDKPHQHWFNRFFEDQETKRSGGKQHSAGLFTLRSAKLTWVPSNKHANASEPWNCYYLNLSCTVDTRLWTQEGTQIVIQEKAAEKAGKLESMRRKENLSKTQQGYIKRLEATLDKLQTPYPRPSRQLYSGKANILAGVSMGLDKPATVAVVDALTGEVLTYRSVKQLLGENHQLLRRAQIEKTKIAHRGHKNRRQGGRKVSEESNVAQQVDRLLAKSIVEIARKYQASSIVVPDLADIREIVETEVKARAQDKVPDFVEGQQQYAKAYRTQVHQWSYRRLQEAVRTKAEQSGITIEVVRQGLSGTQHEKAKALALQGYEKRIREHVEMA

SEQ ID NO: 28 - protein, 537 aa, unidentified
  REGION 1..537: note = Description of Unknown: MG64 effector sequence
  REGION 1..537: note = MG64-41 effector
QLAGQNRWLEVLQNDETLMAVSGLEIQALRAESTKLLDLLSSQITKPAKGSKKTNRGKGKKQAKQTQGKTLYQSLWDLYRETEDILQKCAIAYLLKNKCQVPGKPEDPEKFQHRRRKAEIRAERLNEQLIETRLPKGRDLTNEQWLEALKIATEQVPKDEEEAAIWQSRLLTNAAKFPFPVAYETNEDLKWFLNGKGRLCVRFNGLSEHTFKIYCDQRQLHWFKRFLENKQNKKDNKGKHTSGLFTLRSGRILWKPHSGVVKNAPWTVNHLTLQCSVDTRLWTAEGTEQVRQEKATSIAKVIAGTKAKGNLNRNQLDSISNREKTLELMHNPFPRPSQPIYQGNPSIIAAVSFGLEKPATLAIVDVITGKAITYRSIRQLLGNSYKLFNKQRLKQKQRDYWRHKNQQKSADNRISEGGLGDYVDSLIAKSIVDTAARYEAVSIVLPDQSNIREIIHAEIQAKAERKIPGLKEKQDKYAAQYRRSVHRWSYGRLSQKITTKAAIHGVAIEITRQPLQGTPQEKAVGLAVSAYQSRQVG

SEQ ID NO: 29 - protein, 643 aa, unidentified
  REGION 1..643: note = Description of Unknown: MG64 effector sequence
  REGION 1..643: note = MG64-42 effector
MSQITVQCRLVASEETRQYLWYLMADIYTPFVNEMLRQIREDDNFEQWRQAGKIPAGVFEDYRKALKTESRFQGMPGRWYYAGREEVKRIYKSWLALRRRLRNQLSGQNRWLEVLQSDETLMAVSGLDLPALRAASTKLLDLLSSQIAKPAKGSKKTNRGKGKKQAKQTQGKSLYQSLWDLYKETEDILQKCAIAYLLKNKSQVPDKPEDPEKFRHRRRKAEIRTERLNEQLTKTRLPKGRDLTNEQWLEALAIATEQIPKDETEAAIWQSRLLTDAASLPFPVAYETNEDLKWFLNGKGRLCVSFNGLSEHTFEIYCDKRQLHWFKRFLEDQQIKKEHQGKRSSGLFTLRSGRISWTSPSDIDKSPCWTANRLTLHCSVDTRLWTQEGTEEVRQEKATNIAKIIAGTKAKGNLNQKQQDFITKRETTLKLLHNPFPRPSKPLYQGNPSIIAAVSFGLEKPATLAIVDITTGKAITYRSIRQLLDQNYKLFTKHRLQQQQRAHQRHQNQKESAENRISEGGLGEHVDSLIAKAILETAAEYGASSIVLPELGNIREIIQAEVIAKAERKIPGLKEKQDEYAAKFRASVHRWSYGRLAQKITTKAALQGLETESTRQPLQGSPQEKARNLAIAAYESRKVDQRA

SEQ ID NO: 30 - protein, 526 aa, unidentified
  REGION 1..526: note = Description of Unknown: MG64 effector sequence
  REGION 1..526: note = MG64-43 effector
MPTTLAPKRPLSKSSDYMKTIRCCLCANPETRRYFWKIMVTYTLLVNELLAAMPQRPEFAQWKQRGTIAREAVRIVLTPLKSDPTYADLPKRFFSSAELLVCYVYKSWLALQKRRYWKLVGKQRWLQVIEDDLQSLLTDNFSLESVQSKAHQILEQAHKELEKQPQRFKKKGKKSRPLFGYLLDLYGTTADKLERRAIGHLLHHDLKVSDTEDFPETIQFSIDQQQVEIARLKEQLQSRLPDGRDPTQARFLEKLRIATALPELELEGFDEEHFSEWRTQKQIPLLNPLPYPVLFGSSSDLHWKLEPQKATTEANISPEVPTARSERVKERIQVRFKGDELQDSWFKLQCDRRQLPIFRQFVTDYLCQKQAPDHEKFGEGLFTLRSACLVWKEDPQGARKRKKRRKQGACQDEPWETHRLYLHCTIDTRFLTQEGTEQVRATKLDLAQKALEGIENKTALETVTQEPSAEQQKHLKRKQTTVHRLETQKPPVRPTIQPYEGKSNIVVGVSLSRHEPVTLIVFDTAQ

SEQ ID NO: 31 - skipped

SEQ ID NO: 32 - DNA, 417 bp, synthetic construct
  misc_feature 1..417: note = Description of Artificial Sequence: Synthetic polynucleotide
  misc_feature 1..417: note = MG64-3 effector intergenic region encoding tracrRNA
  source 1..417: mol_type = other DNA
aggttcgtatgtaccactaaaataaatgcagcgcctaagttcatgtcgtcagcggcctctgtgcttagaaaaagggctagtttgactgtctgaacgcagtcttgctttctgacctagataactgtccatccccaaagctgtgagcgcacgcagcaagagggcacgggttccggagtgatggttatcaaattcacctccgagcaaggaggaatccacccaaaacttaaatttggcaaacctaagcgaggtcaaaaaccctgggaggtttgccaaaagactgaagctcctggtctacaaaggtttgagtcatctagtttgtcccaatttctggtctgtcataagaatttagtagaactagattgggctttgccaaattcaactctgcaaagcttgcagggtatgcctttccgatggcaa

SEQ ID NO: 33 - DNA, 468 bp, synthetic construct
  misc_feature 1..468: note = Description of Artificial Sequence: Synthetic polynucleotide
  misc_feature 1..468: note = MG64-5 effector intergenic region encoding tracrRNA
  source 1..468: mol_type = other DNA
ctcagcacagatatctgaaccttgaaaaatgaatatctgatatttcttgtgcgcgccggttctttagggactgagcgataagttagggcgagtttaattgctttccagcccgtgtagttgtccgctctcttgtgcagcttgctgcatgctaggtgtcgggtcgcgccgacatccaagaggccatgtttctgtagttagaggctatctcttcaattatagggatacaggtgtacgtgtcgtggcagctaccaaacagccccgagcaagggggcccatccaaattttggcaaacctcagcgcagtcaatatgcccaggcggtttgccaatctctcaaatccttgtgcaataggtctttcatgcaatctcgtcattgagaagcttcctagaggcgttcggcatccccaaaataattgaggtttgccaaataccccctcgaaaagactgctgtataagcttttcaagctgcg

SEQ ID NO: 34 - DNA, 37 bp, synthetic construct
  misc_feature 1..37: note = Description of Artificial Sequence: Synthetic oligonucleotide
  misc_feature 1..37: note = MG64-3 effector target CRISPR repeat
  source 1..37: mol_type = other DNA
gtcgcccaaggcatttcagggcagggcggattgaaag

SEQ ID NO: 35 - DNA, 37 bp, synthetic construct
  misc_feature 1..37: note = Description of Artificial Sequence: Synthetic oligonucleotide
  misc_feature 1..37: note = MG64-5 effector target CRISPR repeat
  source 1..37: mol_type = other DNA
gtttcatccctgcatttcaatgcagatgggatgaaag

SEQ ID NO: 36 - DNA, 105 bp, synthetic construct
  misc_feature 1..105: note = Description of Artificial Sequence: Synthetic polynucleotide
  misc_feature 1..105: note = MG64-1 LE 105bp
  source 1..105: mol_type = other DNA
tgtacattaacagattatttgtcatcggtaacaaattgttgtcatcttaacaaaatatttgtcatcaataacatattatgtgtcgtgtgcttattactgaaacta

SEQ ID NO: 37 - DNA, 86 bp, synthetic construct
  misc_feature 1..86: note = Description of Artificial Sequence: Synthetic oligonucleotide
  misc_feature 1..86: note = MG64-1 LE 86bp
  source 1..86: mol_type = other DNA
tgtacattaacagattatttgtcatcggtaacaaattgttgtcatcttaacaaaatatttgtcatcaataacatattatgtgtcgt

SEQ ID NO: 38 - DNA, 68 bp, synthetic construct
  misc_feature 1..68: note = Description of Artificial Sequence: Synthetic oligonucleotide
  misc_feature 1..68: note = MG64-1 LE 68bp
  source 1..68: mol_type = other DNA
tgtacattaacagattatttgtcatcggtaacaaattgttgtcatcttaacaaaatatttgtcatcaa

SEQ ID NO: 39 - DNA, 242 bp, synthetic construct
  misc_feature 1..242: note = Description of Artificial Sequence: Synthetic polynucleotide
  misc_feature 1..242: note = MG64-1 RE 242bp
  source 1..242: mol_type = other DNA
tgtacagtaactaattatttgtcgtcttaacaaaatcgtgtcgccgaactatttgagagtgaaagctttatataatcactgctttagctgaattatagcattaatctttgacaaaagttattgcttactatattaacaaattaactgtcattttccagtaaattaacagattaagtgtcatttacccgaatggcacttttttagggactggagcagtattaacaaattacttgtctccaaac

SEQ ID NO: 40 - DNA, 196 bp, synthetic construct
  misc_feature 1..196: note = Description of Artificial Sequence: Synthetic polynucleotide
  misc_feature 1..196: note = MG64-1 RE 196bp
  source 1..196: mol_type = other DNA
tgtacagtaactaattatttgtcgtcttaacaaaatcgtgtcgccgaactatttgagagtgaaagctttatataatcactgctttagctgaattatagcattaatctttgacaaaagttattgcttactatattaacaaattaactgtcattttccagtaaattaacagattaagtgtcatttacccgaatggcac

SEQ ID NO: 41 - DNA, 178 bp, synthetic construct
  misc_feature 1..178: note = Description of Artificial Sequence: Synthetic polynucleotide
  misc_feature 1..178: note = MG64-1 RE 178bp
  source 1..178: mol_type = other DNA
tgtacagtaactaattatttgtcgtcttaacaaaatcgtgtcgccgaactatttgagagtgaaagctttatataatcactgctttagctgaattatagcattaatctttgacaaaagttattgcttactatattaacaaattaactgtcattttccagtaaattaacagattaagtgt

SEQ ID NO: 42 - DNA, 260 bp, synthetic construct
  misc_feature 1..260: note = Description of Artificial Sequence: Synthetic polynucleotide
  misc_feature 1..260: note = MG64-1 RE internal deletion 50
  source 1..260: mol_type = other DNA
tgtacagtaactaattatttgtcgtcttaacaaaatcgtgtcgccgaactatttgagagtgaaagaaagttattgcttactatattaacaaattaactgtcattttccagtaaattaacagattaagtgtcatttacccgaatggcacttttttagggactggagcagtattaacaaattacttgtctccaaacttcaagtattacgatacaattatattaaataaacaattatgtaataatcagacatttttgttcatt

SEQ ID NO: 43 - DNA, 231 bp, synthetic construct
  misc_feature 1..231: note = Description of Artificial Sequence: Synthetic polynucleotide
  misc_feature 1..231: note = MG64-1 RE internal deletion 81
  source 1..231: mol_type = other DNA
tgtacagtaactaattatttgtcgtcttaacaaaatcgtgtcgccgaactatttattaacaaattaactgtcattttccagtaaattaacagattaagtgtcatttacccgaatggcacttttttagggactggagcagtattaacaaattacttgtctccaaacttcaagtattacgatacaattatattaaataaacaattatgtaataatcagacatttttgttcatt

SEQ ID NO: 44 - DNA, 96 bp, synthetic construct
  misc_feature 1..96: note = Description of Artificial Sequence: Synthetic oligonucleotide
  misc_feature 1..96: note = MG64-1 RE internal deletion 81 & 178bp
  source 1..96: mol_type = other DNA
tgtacagtaactaattatttgtcgtcttaacaaaatcgtgtcgccgaactattaacaaattaactgtcattttccagtaaattaacagattaagtg

SEQ ID NO: 45 - DNA, 237 bp, synthetic construct
  misc_feature 1..237: note = Description of Artificial Sequence: Synthetic polynucleotide
  misc_feature 1..237: note = MG64-1 engineered single guide 1
  source 1..237: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcgtgggtttaccacagtgacggctactgaatcacctccgaccaaggaggaatccactgaaaagatggattgaaag

SEQ ID NO: 46 - DNA, 214 bp, synthetic construct
  misc_feature 1..214: note = Description of Artificial Sequence: Synthetic polynucleotide
  misc_feature 1..214: note = MG64-1 engineered single guide 2
  source 1..214: mol_type = other DNA
aaaataatcgcgccgtagatcatgttcttgattgaacctctgaactacgaaaaatgagggttagtttgactctcggcagatagtcttgctttctgaccctagtggctgtccaccctgatgctggaaacagcaataagtggcgtgggtttaccacagtgacggctactgaatcacctccgaccaaggaggaatccactaaaagatggattgaaag

SEQ ID NO: 47 - DNA, 170 bp, synthetic construct
  misc_feature 1..170: note = Description of Artificial Sequence: Synthetic polynucleotide
  misc_feature 1..170: note = MG64-1 engineered single guide 3
  source 1..170: mol_type = other DNA
aaaataatcgcgccgtagatcatgttcttgattgaacctctgaactacgaaaaatgagggttagtttgactctcggcagatagtcttgctttctgaccctagtggctgtgaaaacggctactgaatcacctccgaccaaggaggaatccactgaaaagatggattgaaag

SEQ ID NO: 48 - DNA, 276 bp, synthetic construct
  misc_feature 1..276: note = Description of Artificial Sequence: Synthetic polynucleotide
  misc_feature 1..276: note = MG64-1 engineered single guide 4
  source 1..276: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggatgaaag

SEQ ID NO: 49 - DNA, 281 bp, synthetic construct
  misc_feature 1..281: note = Description of Artificial Sequence: Synthetic polynucleotide
  misc_feature 1..281: note = MG64-1 engineered single guide 5
  source 1..281: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacgaatccactgaaaagatggattgaaag

SEQ ID NO: 50 - DNA, 284 bp, synthetic construct
  misc_feature 1..284: note = Description of Artificial Sequence: Synthetic polynucleotide
  misc_feature 1..284: note = MG64-1 engineered single guide 6
  source 1..284: mol_type = other DNA
gccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 51 - DNA, 283 bp, synthetic construct
  misc_feature 1..283: note = Description of Artificial Sequence: Synthetic polynucleotide
  misc_feature 1..283: note = MG64-1 engineered single guide 7
  source 1..283: mol_type = other DNA
aaaataatcgcgccgtagatcatgcctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 52 - DNA, 273 bp, synthetic construct
  misc_feature 1..273: note = Description of Artificial Sequence: Synthetic polynucleotide
  misc_feature 1..273: note = MG64-1 engineered single guide 8
  source 1..273: mol_type = other DNA
aaaataatcgcgccgtagataactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 53 - DNA, 263 bp, synthetic construct
  misc_feature 1..263: note = Description of Artificial Sequence: Synthetic polynucleotide
  misc_feature 1..263: note = MG64-1 engineered single guide 9
  source 1..263: mol_type = other DNA
aaaataatcgcgccgcgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 54 - DNA, 278 bp, synthetic construct
  misc_feature 1..278: note = Description of Artificial Sequence: Synthetic polynucleotide
  misc_feature 1..278: note = MG64-1 engineered single guide 10
  source 1..278: mol_type = other DNA
aaaataatcgcgccgtagatcatgttcttgattgaacctctgaactacgaaaaatgcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 55 - DNA, 215 bp, synthetic construct
  misc_feature 1..215: note = Description of Artificial Sequence: Synthetic polynucleotide
  misc_feature 1..215: note = MG64-1 engineered single guide 11
  source 1..215: mol_type = other DNA
aaaataatcgcgccgtagatcatgttcttgattgaacctctgaactacgaaaaatgagggttagtttgactctcgcctagtggctgtccaccctgatgctgatttctacatagggtaggtgcgctcccagcaataagtggcgtgggtttaccacagtgacggctactgaatcacctccgaccaaggaggaatccactgaaaagatggattgaaag

SEQ ID NO: 56 - DNA, 221 bp, synthetic construct
  misc_feature 1..221: note = Description of Artificial Sequence: Synthetic polynucleotide
  misc_feature 1..221: note = MG64-1 engineered single guide 12
  source 1..221: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctgtgggtttaccacagtgacggctactgaatcacctccgaccaaggaggaatccactgaaaagatggattgaaag

SEQ ID NO: 57 - DNA, 226 bp, synthetic construct
  misc_feature 1..226: note = Description of Artificial Sequence: Synthetic polynucleotide
  misc_feature 1..226: note = MG64-1 engineered single guide 13
  source 1..226: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcgcagtgacggctactgaatcacctccgaccaaggaggaatccactgaaaagatggattgaaag

SEQ ID NO: 58 - DNA, 224 bp, synthetic construct
  misc_feature 1..224: note = Description of Artificial Sequence: Synthetic polynucleotide
  misc_feature 1..224: note = MG64-1 engineered single guide 14
  source 1..224: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcagtgacggctactgaatcacctccgaccaaggaggaatccactgaaaagatggattgaaag

SEQ ID NO: 59 - DNA, 204 bp, synthetic construct
  misc_feature 1..204: note = Description of Artificial Sequence: Synthetic polynucleotide
  misc_feature 1..204: note = MG64-1 engineered single guide 15
  source 1..204: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcgtgggtttaccacagtgacggctactgaatcacgatgaaag

SEQ ID NO: 60 - DNA, 181 bp, synthetic construct
  misc_feature 1..181: note = Description of Artificial Sequence: Synthetic polynucleotide
  misc_feature 1..181: note = MG64-1 engineered single guide 16
  source 1..181: mol_type = other DNA
gccgtagatcatgcctctgaactacgaaaaatgagggttagtttgactctcggcagatagtcttgctttctgaccctagtggctgtccaccctgatgctgatttctacatagggtaggtgcgctcccagcaataagtggcgtgggtttaccacagtgacggctactgaatcacgatgaaag

SEQ ID NO: 61 - DNA, 262 bp, synthetic construct
  misc_feature 1..262: note = Description of Artificial Sequence: Synthetic polynucleotide
  misc_feature 1..262: note = MG64-1 engineered single guide 17
  source 1..262: mol_type = other DNA
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacgatgaaag

SEQ ID NO: 62 - DNA, 272 bp, synthetic construct
  misc_feature 1..272: note = Description of Artificial Sequence: Synthetic polynucleotide
  misc_feature 1..272: note = MG64-1 engineered single guide 18
  source 1..272: mol_type = other DNA
gccgtagatcatgcctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 63 - DNA, 251 bp, synthetic construct
  misc_feature 1..251: note = Description of Artificial Sequence: Synthetic polynucleotide
  misc_feature 1..251: note = MG64-1 engineered single guide 19
  source 1..251: mol_type = other DNA
gccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacgatgaaag

SEQ ID NO: 64 - protein, 122 aa, unidentified
  REGION 1..122: note = Description of Unknown: MG64 effector sequence
  REGION 1..122: note = MG64-55 effector
MSQITVRARLIAPEETRRAYWDLMAASNTPLINEALRILPTLPDFPKWRQKGNLPDKAAENLIIKLKEDPRFVGQLFWSYISAHKQVTYTFRSWLALQHRKQWKLAGKRLWLEILQPDETLA

SEQ ID NO: 65 - protein, 134 aa, unidentified
  REGION 1..134: note = Description of Unknown: MG64 transposition protein sequence
  REGION 1..134: note = MG64-55-B transposition protein
SSSVSKGTMMAQWRNAYTSILKLIDRQSLKAKSVHLGHLHKGISRARSLREWEAAKKALKKQNKNLTSQNVSTYFEDEERTPQKSMRQRRKAAQKANKKPLPMIEDDLQEESNLEDEKNPLLDLEVTYDDDLFE

SEQ ID NO: 66 - protein, 272 aa, unidentified
  REGION 1..272: note = Description of Unknown: MG64 transposition protein sequence
  REGION 1..272: note = MG64-55-C transposition protein
MEKQVDQIASALGALPELSPEIHKELERLSKRPYILLPKVESCHIFLEECRLGRAHGRIVGDSGVGKTISAKAYSKRLAEASTEKNVIYTILNPNCTPKEFYEKILEALGFTYTKGSIKFLRNRACQVLSRRQISVLFIDEASFLKMDAIGELIYLEESEVVPSIFLIGTDRLDTLLSGNEQVARRYPRYQYGRLHDKELKDVVDLWEQKVLQLPVKSNLKYKAKLNVITKATSGCLGEIDQLLRRAARKALILGESKISLNILREVAGQFE

SEQ ID NO: 67 - protein, 169 aa, unidentified
  REGION 1..169: note = Description of Unknown: MG64 transposition protein sequence
  REGION 1..169: note = MG64-55-Q transposition protein
MNDAQAAQWHFKPEPFEGESFSHFLGRYCAVNCIAPNILAKHIEAGSVAIGRWRKLRYNPSPSERHLQRLADVTGVSQERLLAMLPQEPMQIGTIRLCAACYGEEPCHRIRWQYKSTQFCDRHQLTLLARCPCCKAPFPIPAEWDAGICLRCGKAFVELAEFQKSVLGQ

SEQ ID NO: 68 - RNA, 239 nt, synthetic construct
  misc_feature 1..239: note = Description of Artificial Sequence: Synthetic polynucleotide
  misc_feature 1..239: note = MG64-1 active effector engineered sgRNA 11
  misc_difference 217..239: note = any nucleotide
  source 1..239: mol_type = other RNA
gaaaataatcgcgccgtagatcatgttcttgattgaacctctgaactacgaaaaatgagggttagtttgactctcgcctagtggctgtccaccctgatgctgatttctacatagggtaggtgcgctcccagcaataagtggcgtgggtttaccacagtgacggctactgaatcacctccgaccaaggaggaatccactgaaaagatggattgaaagnnnnnnnnnnnnnnnnnnnnnnn

SEQ ID NO: 69 - RNA, 245 nt, synthetic construct
  misc_feature 1..245: note = Description of Artificial Sequence: Synthetic polynucleotide
  misc_feature 1..245: note = MG64-1 active effector engineered sgRNA 12
  misc_difference 223..245: note = any nucleotide
  source 1..245: mol_type = other RNA
gaaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctgtgggtttaccacagtgacggctactgaatcacctccgaccaaggaggaatccactgaaaagatggattgaaagnnnnnnnnnnnnnnnnnnnnnnn

SEQ ID NO: 70 - RNA, 250 nt, synthetic construct
  misc_feature 1..250: note = Description of Artificial Sequence: Synthetic polynucleotide
  misc_feature 1..250: note = MG64-1 active effector engineered sgRNA 13
  misc_difference 228..250: note = any nucleotide
  source 1..250: mol_type = other RNA
gaaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcgcagtgacggctactgaatcacctccgaccaaggaggaatccactgaaaagatggattgaaagnnnnnnnnnnnnnnnnnnnnnnn

SEQ ID NO: 71 - RNA, 248 nt, synthetic construct
  misc_feature 1..248: note = Description of Artificial Sequence: Synthetic polynucleotide
  misc_feature 1..248: note = MG64-1 active effector engineered sgRNA 14
  misc_difference 226..248: note = any nucleotide
  source 1..248: mol_type = other RNA
gaaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcagtgacggctactgaatcacctccgaccaaggaggaatccactgaaaagatggattgaaagnnnnnnnnnnnnnnnnnnnnnnn

SEQ ID NO: 72 - RNA, 263 nt, synthetic construct
  misc_feature 1..263: note = Description of Artificial Sequence: Synthetic polynucleotide
  misc_feature 1..263: note = MG64-1 active effector engineered sgRNA v2-1
  misc_difference 241..263: note = any nucleotide
  source 1..263: mol_type = other RNA
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccactgaaaggtgggttgaaagnnnnnnnnnnnnnnnnnnnnnnn

SEQ ID NO: 73 - RNA, 262 nt, synthetic construct
  misc_feature 1..262: note = Description of Artificial Sequence: Synthetic polynucleotide
  misc_feature 1..262: note = MG64-1 active effector engineered sgRNA 20
  source 1..262: mol_type = other RNA
  misc_difference 240..262: note = any nucleotide
gccgtagatcatgcctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacgatgaaagnnnnnnnnnnnnnnnnnnnnnnn

SEQ ID NO: 74 - RNA, 241 nt, synthetic construct
  misc_feature 1..241: note = Description of Artificial Sequence: Synthetic polynucleotide
  misc_feature 1..241: note = MG64-3 effector sgRNA
  source 1..241: mol_type = other RNA
  misc_difference 219..241: note = any nucleotide
gaaataaatgcagcgcctaagttcatgtcgtcagcggcctctgtgcttagaaaaagggctagtttgactgtctgaacgcagtcttgctttctgacctagataactgtccatccccaaagctgtgagcgcacgcagcaagagggcacgggttccggagtgatggttatcaaattcacctccgagcaaggaggaatccacccgaaagggcggattgaaagnnnnnnnnnnnnnnnnnnnnnnn

SEQ ID NO: 75 - RNA, 264 nt, synthetic construct
  misc_feature 1..264: note = Description of Artificial Sequence: Synthetic polynucleotide
  misc_feature 1..264: note = MG64-5 effector sgRNA
  misc_difference 242..264: note = any nucleotide
  source 1..264: mol_type = other RNA
gcgcgccggttctttagggactgagcgataagttagggcgagtttaattgctttccagcccgtgtagttgtccgctctcttgtgcagcttgctgcatgctaggtgtcgggtcgcgccgacatccaagaggccatgtttctgtagttagaggctatctcttcaattatagggatacaggtgtacgtgtcgtggcagctaccaaacagccccgagcaagggggcccatccgaaaggatgaaagnnnnnnnnnnnnnnnnnnnnnnn

SEQ ID NO: 76 - DNA, 447 bp, synthetic construct
  misc_feature 1..447: note = Description of Artificial Sequence: Synthetic polynucleotide
  misc_feature 1..447: note = MG64-3 putative transposon end LE
  source 1..447: mol_type = other DNA
aaggcgagtttgggaacggatactcctgggcatttctaactcaacgtggtatcgtcataaatccgagggcgattagcacagtggtagcgcgcttccttcacacggaagaggtcactggttcgaacccagtatcgcccatacatttgtcgaataacgctttatttgtcgtcggtaacacatttgtgtcgtccataacgcttagatgtcatcgataacagttttttgtcaccttctcggtaagtttgccgttaagcgatcgttcacacaatgctgtcgtccaaaattaggcttttgatgtgtgaaggaagcgcgcctttcaatccacctatgcgtatattaacgccaaaacttgatccaagtacaaatgttttaagactgtattctggtctgcatgaaaagctttaggcgtctagatatgagaagccacctaatctacatgcggtcgac

SEQ ID NO: 77 - DNA, 430 bp, synthetic construct
  misc_feature 1..430: note = Description of Artificial Sequence: Synthetic polynucleotide
  misc_feature 1..430: note = MG64-3 putative transposon end RE
  source 1..430: mol_type = other DNA
ggagtcatgcagtacaagtgaattaaatcaaatgtactatgactaccaagatatgtcatctaatttgttagatccgataaagccgccaagaggcgacaaagagtgtgttaatgccaaggtgatctcagattccaagagacgacactgattgtgttcaacttccaagagacgtcagttattctgttagtcaaagagactcattgagatatgagtggaaacgctgaagagtctgatctgaattggttctggctatctcaagctcgatgacaagattgcgttatgacgacattttacgtgttactcgacaacagcggctgacggtggcaccctcagcaattttctcttccacaaagtcgggatcatctgaccccgtgacgaagggccgccagggacgtataccatgtcactgaggcagcaagatagggatcaa

SEQ ID NO: 78 - DNA, 450 bp, synthetic construct
  misc_feature 1..450: note = Description of Artificial Sequence: Synthetic polynucleotide
  misc_feature 1..450: note = MG64-5 putative transposon end LE
  source 1..450: mol_type = other DNA
ttgagacgaaggttgatatctatcgagccgtaactgcttgagctgagttttcaacctctaagcgcaagaaaaaacgactcgtaattcgcgaaaaacgcacatatgaactttcgacttccgatttgcgaaaaatgactttgcgactcgcaatctgcgaatctgtacttgtttactgatttcgggttggagtgccaaaactctctctgggcagggactttacggattatttacaagcgatattaatctgcgaatcgcgacatttaatgtgcgaacgtacaccagatttaaaggattaatttcccaaaaaacacggaagaataggcattttagccgtcaaaccgctattacagtcggtcaattgatgttaaatccgccatttttgccagatttaaggaattagtttccaaaatcctaggccctagttggatgttgggtgcgatcgcagcgctc

SEQ ID NO: 79 - DNA, 583 bp, synthetic construct
  misc_feature 1..583: note = Description of Artificial Sequence: Synthetic polynucleotide
  misc_feature 1..583: note = MG64-5 putative transposon end RE
  source 1..583: mol_type = other DNA
tttagttgtcgcaaagacaactgtaaaagtggtatcacaggtgccctggaatcgtcaaaccctctctattagatcctttttgcttgttttgcggtctgatcaggatcagatttatcgattccaagcttgtcataatgacaattaacgaatagcgatatacgtatacgtacatcgacatgttgatagttgctcacccacacagtggccgcgagcgacttaccgaaaggctcctaggtgtttgtgagtggtattggaaagcagcaaaagcgatcgcaccccctaatggagtctgccgaaacaggcgcaaaacaaagatggcgacgattgcagaaacgcctgacagcagcagccgaggatgggaatactaggctgctaatgtcgatcgcccacaagtacccaaatgcctgaaacccacatcaccgttggagcccatcaagattttgagacttacctgacggaacctttcggtgacgggcctgccagtgatgaacgtgcggccgactttagaaataggctgaaatcagcccagacgctccaagatgctggtttgccaatagtcgaaaatgttttggttggtcgctga

SEQ ID NO: 80 - protein, 803 aa, synthetic construct
  REGION 1..803: note = Description of Artificial Sequence: Synthetic polypeptide
  REGION 1..803: note = MG64-1 effector - MG64-1-Q fusion
MSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSAGGGSGGGSGGGSGGGSGGGSMESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYL

SEQ ID NO: 81 - protein, 803 aa, synthetic construct
  REGION 1..803: note = Description of Artificial Sequence: Synthetic polypeptide
  REGION 1..803: note = MG64-1-Q - MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 82 - protein, 831 aa, synthetic construct
  REGION 1..831: note = Description of Artificial Sequence: Synthetic polypeptide
  REGION 1..831: note = MG64-1-Q- 48aa Linker- MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGGGSGGGSGGGSGGGSGGGSGGGSGGGSGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 83 - protein, 851 aa, synthetic construct
  REGION 1..851: note = Description of Artificial Sequence: Synthetic polypeptide
  REGION 1..851: note = MG64-1-Q- 68aa Linker- MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGGGSGGGSGGGSGGGSGGGSGGGSGGGSGGGSGGGSGGGSGGGSGGGSGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 84 - protein, 855 aa, synthetic construct
  REGION 1..855: note = Description of Artificial Sequence: Synthetic polypeptide
  REGION 1..855: note = MG64-1-Q- 72aa Linker- MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGGGSGGGSGGGSGGGSGGGSGGGSGGGSGGGSGGGSGGGSGGGSGGGSGGGSGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 85 - protein, 860 aa, synthetic construct
  REGION 1..860: note = Description of Artificial Sequence: Synthetic polypeptide
  REGION 1..860: note = MG64-1-Q- 77aa Linker- MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGTKYLGGGSGGGSGGGSGGGSGGGSGGGSGGGSGGGSGGGSGGGSGGGSGGGSGGGSGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 86 - protein, 16 aa, synthetic construct
  REGION 1..16: note = Description of Artificial Sequence: Synthetic peptide
  REGION 1..16: note = Nucleoplasmin NLS
KRPAATKKAGQAKKKK

SEQ ID NO: 87 - protein, 19 aa, synthetic construct
  REGION 1..19: note = Description of Artificial Sequence: Synthetic peptide
  REGION 1..19: note = SV40 2x NLS
PKKKRKVDGSPKKKRKVDS

SEQ ID NO: 88 - protein, 19 aa, synthetic construct
  REGION 1..19: note = Description of Artificial Sequence: Synthetic peptide
  REGION 1..19: note = P2A
ATNFSLLKQAGDVEENPGP

SEQ ID NO: 89 - protein, 18 aa, synthetic construct
  REGION 1..18: note = Description of Artificial Sequence: Synthetic peptide
  REGION 1..18: note = T2A
EGRGSLLTCGDVEENPGP

SEQ ID NO: 90 - protein, 9 aa, synthetic construct
  REGION 1..9: note = Description of Artificial Sequence: Synthetic peptide
  REGION 1..9: note = HA
YPYDVPDYA

SEQ ID NO: 91 - protein, 10 aa, synthetic construct
  REGION 1..10: note = Description of Artificial Sequence: Synthetic peptide
  REGION 1..10: note = Myc
EQKLISEEDL

SEQ ID NO: 92 - protein, 8 aa, synthetic construct
  REGION 1..8: note = Description of Artificial Sequence: Synthetic peptide
  REGION 1..8: note = FLAG
DYKDDDDK

SEQ ID NO: 93 - DNA, 486 bp, synthetic construct
  misc_feature 1..486: note = Description of Artificial Sequence: Synthetic polynucleotide
  misc_feature 1..486: note = MG64-1 active transposon end LE
  source 1..486: mol_type = other DNA
ctggctaatgcacccagtaaggcagcggtatcatcaacggggtctgacgctcagtggaacgaaaactcacgttaggctctcttaaggaaatctatgtaagtttgttgggttagttgcgttttcagtaaatactgtgttatagtaagaacttgtgcggacgtatagctcagttggttagagtacatcgttgacatcgatggggtcactggttcgagtccagttacgtccatatttttttgaagtgtgtataatattaactatgtgactttatgtacattaacagattatttgtcatcggtaacaaattgttgtcatcttaacaaaatatttgtcatcaataacatattatgtgtcgtgtgcttattactgaaactaatcctagacgatggtaaaaaatagaacacatttccccgaaaagtgccacctgacgtctaagaaaccattattatcatgacattaacctataaaaataggcgtatcacgagg

SEQ ID NO: 94 - DNA, 199 bp, synthetic construct
  misc_feature 1..199: note = Description of Artificial Sequence: Synthetic polynucleotide
  misc_feature 1..199: note = MG64-3 effector putative tracrRNA
  source 1..199: mol_type = other DNA
tgcagcgcctaagttcatgtcgtcagcggcctctgtgcttagaaaaagggctagtttgactgtctgaacgcagtcttgctttctgacctagataactgtccatccccaaagctgtgagcgcacgcagcaagagggcacgggttccggagtgatggttatcaaattcacctccgagcaaggaggaatccacccaaaactt

SEQ ID NO: 95 - DNA, 237 bp, synthetic construct
  misc_feature 1..237: note = Description of Artificial Sequence: Synthetic polynucleotide
  misc_feature 1..237: note = MG64-5 effector putative tracrRNA
  source 1..237: mol_type = other DNA
tcttgtgcgcgccggttctttagggactgagcgataagttagggcgagtttaattgctttccagcccgtgtagttgtccgctctcttgtgcagcttgctgcatgctaggtgtcgggtcgcgccgacatccaagaggccatgtttctgtagttagaggctatctcttcaattatagggatacaggtgtacgtgtcgtggcagctaccaaacagccccgagcaagggggcccatccaaa

SEQ ID NO: 96 - RNA, 239 nt, synthetic construct; identical to SEQ ID NO: 68
  misc_feature 1..239: note = Description of Artificial Sequence: Synthetic polynucleotide
  misc_feature 1..239: note = MG64-1 active effector engineered sgRNA 11
  misc_difference 217..239: note = any nucleotide
  source 1..239: mol_type = other RNA

SEQ ID NO: 97 - RNA, 245 nt, synthetic construct; identical to SEQ ID NO: 69
  misc_feature 1..245: note = Description of Artificial Sequence: Synthetic polynucleotide
  misc_feature 1..245: note = MG64-1 active effector engineered sgRNA 12
  source 1..245: mol_type = other RNA
  misc_difference 223..245: note = any nucleotide

SEQ ID NO: 98 - RNA, 250 nt, synthetic construct; identical to SEQ ID NO: 70
  misc_feature 1..250: note = Description of Artificial Sequence: Synthetic polynucleotide
  misc_feature 1..250: note = MG64-1 active effector engineered sgRNA 13
  misc_difference 228..250: note = any nucleotide
  source 1..250: mol_type = other RNA

SEQ ID NO: 99 - RNA, 248 nt, synthetic construct; identical to SEQ ID NO: 71
  misc_feature 1..248: note = Description of Artificial Sequence: Synthetic polynucleotide
  misc_feature 1..248: note = MG64-1 active effector engineered sgRNA 14
  misc_difference 226..248: note = any nucleotide
  source 1..248: mol_type = other RNA

SEQ ID NO: 100 - RNA, 263 nt, synthetic construct; identical to SEQ ID NO: 72
  misc_feature 1..263: note = Description of Artificial Sequence: Synthetic polynucleotide
  misc_feature 1..263: note = MG64-1 active effector engineered sgRNA v2-1
  misc_difference 241..263: note = any nucleotide
  source 1..263: mol_type = other RNA

SEQ ID NO: 101 - RNA, 262 nt, synthetic construct; identical to SEQ ID NO: 73
  misc_feature 1..262: note = Description of Artificial Sequence: Synthetic polynucleotide
  misc_feature 1..262: note = MG64-1 active effector engineered sgRNA 20
  misc_difference 240..262: note = any nucleotide
  source 1..262: mol_type = other RNA

SEQ ID NO: 102 - RNA, 241 nt, synthetic construct; identical to SEQ ID NO: 74
  misc_feature 1..241: note = Description of Artificial Sequence: Synthetic polynucleotide
  misc_feature 1..241: note = MG64-3 effector sgRNA
  misc_difference 219..241: note = any nucleotide
  source 1..241: mol_type = other RNA

SEQ ID NO: 103 - RNA, 264 nt, synthetic construct; identical to SEQ ID NO: 75
  misc_feature 1..264: note = Description of Artificial Sequence: Synthetic polynucleotide
  misc_feature 1..264: note = MG64-5 effector sgRNA
  misc_difference 242..264: note = any nucleotide
  source 1..264: mol_type = other RNA

SEQ ID NO: 104 - RNA, 199 nt, synthetic construct
  misc_feature 1..199: note = Description of Artificial Sequence: Synthetic polynucleotide
  misc_feature 1..199: note = MG64-3 effector putative tracrRNA sequence
  source 1..199: mol_type = other RNA
tgcagcgcctaagttcatgtcgtcagcggcctctgtgcttagaaaaagggctagtttgactgtctgaacgcagtcttgctttctgacctagataactgtccatccccaaagctgtgagcgcacgcagcaagagggcacgggttccggagtgatggttatcaaattcacctccgagcaaggaggaatccacccaaaactt

SEQ ID NO: 105 - RNA, 237 nt, synthetic construct
  misc_feature 1..237: note = Description of Artificial Sequence: Synthetic polynucleotide
  misc_feature 1..237: note = MG64-5 effector putative tracrRNA sequence
  source 1..237: mol_type = other RNA
tcttgtgcgcgccggttctttagggactgagcgataagttagggcgagtttaattgctttccagcccgtgtagttgtccgctctcttgtgcagcttgctgcatgctaggtgtcgggtcgcgccgacatccaagaggccatgtttctgtagttagaggctatctcttcaattatagggatacaggtgtacgtgtcgtggcagctaccaaacagccccgagcaagggggcccatccaaa

SEQ ID NO: 106 - RNA, 38 nt, synthetic construct
  misc_feature 1..38: note = Description of Artificial Sequence: Synthetic oligonucleotide
  misc_feature 1..38: note = MG64-1 active effector's crRNA sequence
  source 1..38: mol_type = other RNA
cgtcacaatctattttggttaatgagatggattgaaag

SEQ ID NO: 107 - RNA, 37 nt, synthetic construct
  misc_feature 1..37: note = Description of Artificial Sequence: Synthetic oligonucleotide
  misc_feature 1..37: note = MG64-3 effector crRNA sequence
  source 1..37: mol_type = other RNA
gtcgcccaaggcatttcagggcagggcggattgaaag

SEQ ID NO: 108 - RNA, 37 nt, synthetic construct
  misc_feature 1..37: note = Description of Artificial Sequence: Synthetic oligonucleotide
  misc_feature 1..37: note = MG64-5 effector crRNA sequence
  source 1..37: mol_type = other RNA
gtttcatccctgcatttcaatgcagatgggatgaaag